(19) United States
(12) Patent Application Publication
(10) Pub. No.: US 2024/0342070 A1
(43) Pub. Date: Oct. 17, 2024

(54) Topical Compositions and Formulations with Multifunctional Actions, Including Activity Against Parasites

(71) Applicant: BIOSAFE TECHNOLOGIES, INC., Denison, TX (US)

(72) Inventor: Lynn James-Meyer, Denison, TX (US)

(73) Assignee: BIOSAFE TECHNOLOGIES, INC., Denison, TX (US)

(21) Appl. No.: 18/682,911

(22) PCT Filed: Aug. 10, 2022
(86) PCT No.: PCT/US22/40009

§ 371 Date: Feb. 10, 2024

Related U.S. Application Data
(60) Provisional application No. 63/231,717, filed on Aug. 10, 2021.

(51) Int. Cl. A61K 8/60; A61K 8/34; A61K 8/365; A61K 8/39; A61K 8/44; A61Q 5/00; A61Q 17/00; A61Q 19/00

(52) U.S. Cl. CPC A61K 8/604; A61K 8/345; A61K 8/365; A61K 8/39; A61K 8/44; A61Q 5/00; A61Q 17/005; A61Q 19/00

(57) ABSTRACT

Compositions and/or formulations and products thereof for hair and/or skin are disclosed, which have a multi-functional effect that includes at least two or more functions, or three or more functions, or four or more functions, the functions selected from anti-parasitic, prophylactic or preventative, cleansing, and conditioning. The compositions and/or formulations for hair and/or skin may utilize at least one alkyl polyglucoside (APG) as an active component and a primary surfactant, in an unexpected amount. In addition to the APG as a primary surfactant, additional components or ingredients include: water, at least one co-solvent for water, a pH balancing agent, and at least one separate antimicrobial or preservative. The compositions and/or formulations are further prepared with at least two or more of: at least one humectant, at least one chelating agent, at least one solubilizing and clarifying agent, and/or at least one conditioning agent. In some embodiments, a humectant may be replaced by a ceramide or by an emollient (as a natural oil or natural butter).

### CROSS REFERENCE TO RELATED APPLICATIONS

This application is a national phase patent application from International Application No. PCT/US2022/040009 that entered the U.S. on Feb. 10, 2024, as U.S. patent application Ser. No. 18/682,911, in which International Application PCT/US2022/040009 claims the benefit of priority to U.S. Provisional Application No. 63/231,717 filed Aug. 10, 2021, the contents of which are herein incorporated by reference to the full extent allowable and available.

### BACKGROUND

Parasitic infestation remains a global concern. Parasites include invertebrates that bite or feed on or are in hair and/or skin (e.g., animal or human skin), such as lice, biting flies, fleas, bedbugs, mites, scabies, and ticks. Infestation may include activity by one or a number of parasites, the number of parasites being the same type of parasite, or different types and/or species of parasites.

Issues related to the biting or feeding of one or a number of parasites include concerns regarding harmful effects, such as illness and/or infection. For example, skin parasites leave swellings after biting and/or feeding. The swellings or inflammation may be accompanied by red papules or welts, as well as negative symptoms, such as pain and itchiness. Because human fingernails contain many bacteria, itching and scratching of papules introduce bacteria, which can result in infection (secondary infection), and, in some instances, physiologic, hormonal, and/or neurologic syndromes. In addition, some parasites carry disease-causing pathogens or microorganisms, such as viruses and/or bacteria. Many current treatments against parasites are ineffective, or have only limited or short-term effect or temporarily reduce certain symptoms, but not necessarily the underlying issue, secondary infection, etc.

Lice, or ectoparasites, feed on blood; the feeding occurs in all life stages of the ectoparasite. With biting or feeding, or shortly thereafter, papules or welts will form on skin. Large infestations can produce rashes with or without black spots (blood filled fecal pellets). Some lice have been found to transmit disease-causing pathogens. Some insecticide shampoo treatments for lice have led to resistance to the insecticide in the population of louse. Fleas feed on blood when in an adult stage and with biting or feeding, or shortly thereafter, papules or welts form on the skin; the bites may become swollen and inflamed. Some fleas are known to transmit disease-causing pathogens, such as bacteria that cause the plague. There are also allergies, sometimes severe, that can develop in response to biting and/or feeding from fleas. Some treatments for fleas have led to resistance to the insecticide in the population of fleas.

Flies, as biting and/or feeding flies, include but are not limited to mosquitoes (family Culicidae), black flies (family Simuliidae), biting gnats (family Ceratopogonidae), sand flies (family Psychodidae), snipe flies (family Rhagionidae), horse and deer flies (family Tabanidae) and stable flies (family Muscidae), which feed on blood. With biting or feeding, or shortly thereafter, raised papules or welts will form on the skin. Some of these flies have been found to transmit disease-causing pathogen(s) upon biting or feeding. Other flies, such as human bot fly (family Oestridae), and bowflies (family Calliphoridae) have larval stages that live on or in skin. Some current treatments against biting and/or feeding flies have led to resistance to the insecticide in the population of flies.

Bedbugs feed on blood at all life stages; with biting or feeding, or shortly thereafter, red papules or welts form on the skin. Allergic hypersensitivity can result from bedbug bites and/or feeding. Some treatments for bedbugs have led to resistance to the insecticide in the population of bedbugs. Mites and scabies feed on blood; some at adult stage, others at all life stages and with biting or feeding, or shortly thereafter, papules, itchy irritations, or a rash form on skin, while scabies can burrow into the skin. Allergic hypersensitivity or contact dermatitis can result from bites and/or feeding. Some treatments for scabies have led to resistance to the insecticide used in the population of bedbugs. Ticks feed on blood, generally at adult stage; with biting or feeding, or shortly thereafter, red papules or welts, and/or a rash or blisters will form on the skin. Some ticks are known to transmit disease-causing pathogens, such as Lyme disease. Some antibiotic or insecticide treatments for ticks have led to resistance to the antibiotic or insecticide in the population of ticks.

There remains a need for new and/or improved compositions and/or formulations that reduce, minimize, and/or treat an infestation from one or a number of parasites, the parasite being one type of biting and/or feeding parasite or a number of different types of biting and/or feeding parasites, selected from at least one or more of lice, biting flies, fleas, bedbugs, mites and ticks.

There remains a need for one or more active components that, when provided in a unique and unexpected composition and/or formulation, are anti-parasitic, and said composition and/or formulation is a unique and unexpected composition or formulation with anti-parasitic properties against at least one or more of lice, biting flies, fleas, bedbugs, mites and ticks.

There remains a need for one or more unique and unexpected composition and/or formulation having anti-parasitic properties that are easy to use, and are safe on hair and/or skin while being effective against at least one or more of lice, biting flies, fleas, bedbugs, mites and ticks.

There remains a need for one or more unique and unexpected composition and/or formulation having anti-parasitic properties that are environmentally friendly, containing components that are both safe and effective for use on hair and/or skin against a diverse and varied population or parasites, including at least one or more of lice, biting flies, fleas, bedbugs, mites, scabies, and ticks.

There remains a need for one or more unique and unexpected compositions and/or formulations having anti-parasitic properties, including a killing and/or knock down effect against at least one or more of lice, biting flies, fleas, bedbugs, mites and ticks, and that contain a status and/or notification as Generally Recognized As Safe (GRAS) (e.g., in compliance with or in association with U.S. Food and Drug Administration).

There is also a need for one or more unique and unexpected compositions and/or formulations having not only anti-parasitic properties (e.g., killing and/or knock down effect against one or more parasites being at least one or more of lice, biting flies, fleas, bedbugs, mites and ticks), are also utilized for prevention against a further infestation of the one or more parasites.

There is also a need for one or more unique and unexpected compositions and/or formulations having not only anti-parasitic properties (e.g., killing and/or knock down effect against one or more parasites being at least one or more of lice, biting flies, fleas, bedbugs, mites and ticks), and preventative utility, but are also suitable for use on hair and/or skin as a true shampoo and/or conditioner, leaving hair and/or skin clean (e.g., for removal of unwanted sebum, dandruff, environmental dirt and dust, other residues) as well as leaving conditioning components onto hair (e.g., for protection, moisturizing, removing tangles, increase elasticity and increase shine).

Parasite infestation (including fleas and lice), particularly in children, are on the rise in the US, UK, Europe, as well as in most under-developed or less developed countries. Because of an increase in resistance and many safety concerns with insecticide-based treatments, there is a demand for safe parasite treatments having a physical or chemical mode of action with a killing and/or knock down effect. These and other described needs are met by inventive compositions and/or formulations herein having a unique formula, most containing an unexpected quantity of at least one alkyl polyglucoside (APG).

Alkyl polyglucosides (APGs) are additives having both hydrophobic and hydrophilic regions. The hydrophilic group includes a glucoside (e.g., D-glucose or saccharide unit), often from one to five units. The hydrophobic group is a fatty alkyl chain, linear or branched, generally having a chain length from about five or six carbon atoms to about eighteen or twenty carbon atoms. In general, the shorter the alkyl chain length is, the lower the surface activity of the alkyl polyglucoside. Formed from condensation of a fatty alcohol (or mixtures having varying chain lengths) and a glucose, when plant-based, the APG is biodegradable and does not have to be recycled. Some plant sources for the fatty alkyl chain are vegetable oils, such as corn, or palm kernel oil, as well as coconut oil, to name a few. Other plant source may be used as is understood by one skilled in this field and art.

Biodegradability of certain APGs has made them attractive as a surface-active agent or surfactant. APGs are utilized as a secondary surfactant in a shampoo to enhance foaming activity of a primary surfactant. Their foaming action and synergy with a primary surfactant has led to inclusion of APGs in low quantities as a secondary surfactant in personal care products, such as skin or hair cleansing products (rinse-off products, such as, shampoo, shower gel, hand wash, spray or pump cleanser, as representative examples), therefore being included at quite low concentrations. However, APGs behave differently at differing concentrations. In typical use, in a low quantity, it is a non-ionic surfactant, provided as and behaving as a weak emulsifier and/or foaming agent. APGs are diluted prior to use in a shampoo due to its very high viscosity and very high pH, so that its working quantity as a secondary surfactant in ready-to-use hair or skin cleaning products (rinse-off products) is below 10% based on the wt. % of an entire composition in which the APG is utilized in, and is typically in a low amount (e.g., less than about 7% or less than about 6% or less than about 5% or less than about 4% or less than about 3%, based on the wt. % of a composition in which the APG is utilized in).

Introduction of an APG, as a secondary surfactant in a working or ready-to-use skin or hair cleansing product produce micelles at low concentrations. APG behaves differently above the critical micelle concentration, forming more of a thick monolayer that can split, which is why APG is not utilized at higher amounts. APGs form lyotropic liquid crystals or precipitates at higher concentrations (often at or greater than about 15%, or at or greater than about 20%, or at or greater than about 25%, or at or greater than about 30% and above). This behavior, in combination with their high viscosity and pH means that APGs have not been found to be useful at higher concentrations (e.g., when in a working amount that is greater than about 12%, based on the total weight of the working or ready-to-use composition or product), and APGs are not provided in hair or skin cleansing or conditioning products at the higher concentrations. In view of many issues to overcome when utilizing higher concentrations of APG, there is a need to provide one or more unique and unexpected compositions and/or formulations that achieve stability of an APG at a high concentration and in a lower pH, and maintains handleability and split suppression, particularly during storage.

### SUMMARY

Described herein are novel and previously unreported compositions and/or new uses of said compositions that meet at least one or more than one of the needs or issues described above.

In one or more embodiments, described herein are one or more unique and unexpected compositions and/or formulations for utility on hair and/or skin, each having more than one function, including having anti-parasitic properties (e.g., killing and/or knock down effect against a parasite being one or more of lice, biting flies, fleas, bedbugs, mites and ticks) by a chemical or physical mode of action.

In one or more embodiments, the one or more unique and unexpected compositions and/or formulations herein, when utilized on hair and/or skin, will kill and/or knock down one or more parasites (being one or more of lice, biting flies, fleas, bedbugs, mites, and ticks in hair and/or on skin), the killing and/or knock down effect being sufficient to kill and/or knock down the one or more parasites after a single use with an efficiency (efficacy) of the compositions and/or formulations considered as up to or greater than 90% efficient (effective), or up to or greater than 95% efficient (effective), or up to or about 100% efficient (effective), or close to 100% efficient (effective). In addition, or as an alternative, in one or more embodiments, the one or more unique and unexpected compositions and/or formulations is a preventative product, to prevent re-infestation of the one or more parasites in hair and/or on skin.

In one or more embodiments, described herein are one or more unique and unexpected compositions and/or formulations that have a plurality of effects on hair and/or skin after a single use, wherein the effects include: (i) sufficient killing and/or knock down effect against one or more parasites being at least one or more of lice, biting flies, fleas, bedbugs, mites, scabies, and ticks in order to achieve sufficient removal of the one or more parasites from the hair and/or skin; (ii) sufficient preventative effect in order to prevent immediate re-infestation on hair and/or skin of one or more parasites being at least one or more of lice, biting flies, fleas, bedbugs, mites and ticks; (iii) sufficient cleaning effect to clean hair and/or skin by performing at least one or more of removal of unwanted sebum, removal of dandruff, removal of environmental dirt and dust, and removal of residues from hair and/or skin; and (iv) sufficient conditioning effect to condition hair and/or skin by leaving conditioning components onto hair and/or skin for one or more functions, the functions being any one or more of protection, moisturizing, removing tangles, increase elasticity and increasing shine. In some embodiments, the plurality of effects are at least two effects, such as (i) and (ii), or (i) and (iii), or (i) and (iv), or (ii) and (iii), or (ii) and (iv), or (iii) and (iv). In some embodiments, the plurality of effects may be at least three effects, such as (i) and (ii) and (iii), or (i) and (ii) and (iv), or (ii) and (iii) and (iv) or (i) and (iii) and (iv). In some embodiments, the plurality of effects are at least four effects: (i) and (ii) and (iii) and (iv). The herein described compositions and/or formulations and products thereof for hair and/or skin have a multi-functional effect that includes at least two or more functions, or three or more functions, or four or more functions, the functions selected from two or more of an insecticide that is anti-parasitic (i), prophylaxis or prevention of re-infestation (ii), hair and/or skin cleansing (iii), and hair and/or skin conditioning (iv).

In one or more embodiments, compositions and/or formulations for hair and/or skin include and utilize at least one APG as an active component (e.g., for anti-parasitic activity and/or prophylactic or preventative action), the APG being a primary surfactant in each composition and/or formulation and in an unexpected amount, with unexpected stability of the APG at its high concentration and lower pH, such compositions having good handleability, split suppression, and stability with long-term storage. Importantly, a novel finding herein is that an APG in a quantity and composition/formulation herein is an insecticide that kills and/or knocks down parasites (at least one selected from a group consisting of lice, biting flies, fleas, bedbugs, mites, scabies and ticks), and kills and/or allows for removal of eggs and nits. In addition to the at least one APG, each composition and/or formulation for hair and/or skin will include the following ingredients or components: water, at least one co-solvent for water, a pH balancing agent, and at least one separate antimicrobial and/or preservative. In one or more embodiments, compositions and/or formulations are further prepared with two or more of: at least one humectant, at least one chelating agent, at least one solubilizing and clarifying agent, and/or at least one conditioning agent. Selection of one or more APG (from APGs disclosed herein) may be utilized to tailor make and provide an insecticide for activity against a particular parasite or combination of parasites.

In one or more embodiments is a composition and/or formulation (or product therefrom) for hair and/or skin comprising a uniform blend, and the uniform blend includes at least the following ingredients: an APG as a primary surfactant in the composition and/or formulation; filtered water; a co-solvent for the filtered water; a humectant; a chelating agent; a pH balancing agent in a sufficient amount to adjust pH to between about 7.4 and greater than 6.0 (any pH therein); a solubilizing and clarifying agent; and an antimicrobial agent, wherein the antimicrobial agent may also act as a preservative. For such a composition and/or formulation, the ingredients/components, when combined in described amounts, cause and ensure stability of the blend as well as handleability and suppress splitting. The APG as a primary surfactant may be selected from, as non-limiting examples, one or more of: lauryl glucoside; a C12 alkyl glucoside; a C10-C14 alkyl glucoside; a coco glucoside that is a coconut based glucoside with any combination of C8-C16 alkyl glucosides, wherein greater than 50% of total alkyl glucosides are one or more of C12 alkyl glucosides and C14 alkyl glucosides; a coconut based glucoside with about 40% C8-C10 alkyl glucosides and about 60% C12-C16 alkyl glucosides; a decyl glucoside having about 60% C8-C10 alkyl glucosides and about 40% C12-C14 alkyl glucosides; and dodecyl D-glucoside having a combination of about 40% C8-C10 alkyl glucosides and about 60% C12-C14 alkyl glucosides. Further examples are provided elsewhere herein. Without a thickener, the uniform blend may be provided as a shampoo, mousse, spray and/or spritz; an added thickener allows for a cream, balm and/or paste.

The APG is in an amount greater than about 13% or 14% or 15% and less than about 40%, based on a weight of a total composition, in a ready-to-use composition or formulation. The humectant may be selected from, as non-limiting examples, one or more of glycerin, glyceryl stearate, triglyceryl stearate, sorbitol, butylene glycol, diethylene glycol, 1,3-propanediol (plant-based alternative to propylene glycol), caprylyl glycol, glycine betaine, grape seed oil, lanolin, acetylated lanolin, honey, pyroglutamic acid, and tristearin. Further examples are provided elsewhere herein. The humectant may be in an amount between about 0.5% and about 3%, based on weight of a total composition. The chelating agent may be selected from, as non-limiting examples, one or more of disodium ethylene diamine tetraacetic acid (EDTA), disodium EDTA-copper, HEDTA, oxalic acid, potassium citrate, sodium citrate, tetrahydroxypropyl ethylenediamine, sodium oxalate, TEA-EDTA, tetrasodium EDTA, trisodium EDTA, trisodium HEDTA, nitrilotriacetic acid (NTA), iminodisuccinate (IDS), citric acid, sodium gluconate, ethylenediamine-N,-N-disuccinic acid (EDDA), ethylenediaminedisuccinic acid (EDDS), L-glutamic acid N,N-diacetic acid tetrasodium salt (GLDA), methyl glycinediacetic acid (MGDA), ethylenediamine N,N′-diglutaric acid (EDDG), ethylenediamine-N,N-diamalonic acid (EDDM), 2-hydroxyethyliminodiacetic acid (HEIDA), 3-hydroxy-2,2-iminodisuccinic acid (HIDS), and pyridine-2,6-dicarboxylic acid (PDA). Further examples are provided elsewhere herein. The chelating agent may be in an amount between about 0.05% and about 1%, based on weight of a total composition. The co-solvent for water may be selected from, as non-limiting examples, one or more of glycol, a modified glycol, and polyethylene glycol. Further examples are provided elsewhere herein. The co-solvent for the water may be in an amount between about 5% and about 15%, based on a weight of a total composition. The pH balancing agent may be selected from, as non-limiting examples, one or more of citric acid, salicylic acid, phytic acid, and lactic acid. Further examples are provided elsewhere herein. The pH balancing agent may be in an amount between about 0.1% and about 2%, based on weight of a total composition. The solubilizing and clarifying (SAC) agent may be more than one, such as a first SAC agent and a second SAC agent, wherein the first SAC agent, when included, may be selected from, as non-limiting examples, one or more of polysorbate 20, polysorbate 80, a fatty acid alkanolamide, lauric monoethanolamide, diethanolamide, cocamide, decyl glucoside, coco glucoside, caprylyl/capryl glucoside, polyethylene glycol (PEG)-50 shea butter, olive oil PEG-7 esters, coco betaine, and sulfated castor oil, and wherein the second SAC, when included, may be selected from, as non-limiting examples, one or more of a fatty alkanolamide or an alkyl taurate amides or taurate salt; the alkyl taurate amides or taurate salt may include sodium methyl cocoyl taurate, potassium taurate, sodium methyl taurate, sodium taurate, sodium lauroyl taurate, calcium lauroyl taurate, sodium methyl lauroyl taurate. Further examples are provided elsewhere herein. The SAC agent may be in an amount between about 1% and 8%, based on weight of a total composition. The antimicrobial/preservative may be selected from, as non-limiting examples, one or more from a group consisting of glycol ether, caprylyl glycol, isothiazolinones, citricidals, isopentyldiol, phenoxyethanol, stannic chloride, EDTA, benzoic acid/sodium benzoate, sorbic acid/potassium sorbate, levulinic acid, anisic acid, benzalkonium chloride, triclosan, and ethyl lauroyl arginate. Further examples are provided elsewhere herein. The antimicrobial/preservative may be in an amount between about 0.1% and about 1%, based on weight of a total composition. The filtered water may be in an amount between about 35% and about 65% of the composition (or to 100%), based on weight of a total composition, especially as a ready-to-use composition.

In one or more embodiments is a composition and/or formulation (or product therefrom) for hair and/or skin in which the composition comprises a uniform or homogenous blend (utilizing a proprietary procedure of mixing, heating, and cooling, in which pH is adjusted at the end with a pH balancer). The uniform or homogenous blend includes at least the following ingredients: an APG as a primary surfactant in the composition, wherein the APG may be at least a lauryl glucoside or a combination of a lauryl glucoside with another APG described elsewhere herein; filtered water; a co-solvent for the water, wherein the co-solvent for the water may be polyethylene glycol with a low average number of ethylene glycol units that is at or about or up to about 400 ethylene glycol units; a humectant, wherein the humectant may be a vegetable glycerin, diethylene glycol, or 1,3-propanediol (a plant-based alternative to propylene glycol); a chelating agent, wherein the chelating agent may be glutamic acid, N,N-diacetic acid, tetrasodium salt; a pH balancing agent in a sufficient amount to adjust pH of the composition to a pH less than about 7.4 and greater than about 6.0, wherein the pH balancing agent may be citric acid; a SAC agent, wherein the SAC agent may be a first SAC agent and a second SAC agent, wherein the first SAC agent may be polysorbate 20 and/or a fatty alkanolamide and/or decyl glucoside, and the second SAC agent, if used, may be a fatty alkanolamide and/or a sodium methyl cocoyl taurate; and an antimicrobial agent, wherein the antimicrobial agent is also a preservative, and the antimicrobial agent may be one a combination of one or more antimicrobial agents selected from one or more of caprylyl glycol, phenoxyethanol, and isopentyldiol. For such a composition and/or formulation as a ready-to-use product, the APG may be between about 25% and about 35% of a total composition. The co-solvent for water may be between about 9% and about 13% of a total composition. The chelating agent may be between about 0.1% and about 0.9% of a total composition. The humectant may be between about 1% and about 3% of a total composition. The antimicrobial agent may be between about 0.1% and about 0.9% of a total composition. The SAC agent may be in an amount that is between about 2% and about 6% of a total composition. The filtered water may be between about 40% and about 65% of a total composition. Without a thickener, the ready-to-use uniform blend may be provided as a shampoo, mousse, spray and/or spritz. An added thickener allows for a cream, balm and/or paste to be prepared and utilized.

In some embodiments, a ceramide may replace a humectant in amounts described for the humectant; thus, a humectant may be replaced by a ceramide. In some embodiments, an emollient (natural oil, natural fatty acid, natural butter) may replace a humectant in amounts described for the humectant; thus, a humectant may be replaced by an emollient as a natural oil, fatty acid, or butter. In some embodiments, at least one thickening agent (preferably a natural fatty acid, natural butter, natural starch, natural gel, may be included to provide a thicker product (e.g., as a cream, paste, or balm).

In one or more embodiments, the composition may further comprise (optionally) one or more essential oils, particularly ones that may repel an insect. In one or more embodiments, the composition may further comprise (optionally) one or more skincare or haircare enzymes, including coenzyme Q10, superoxide dismutase (with or without catalase), a protease, diacylglyceroltransferase (e.g., lysyl or prolyl hydroxylase with or without Vitamin C), peroxidase, fruit enzymes (e.g., bromelain, papain), as non-limiting examples. In one or more embodiments, the composition may further comprise (optionally) one or more natural pigments or natural colorants. Inclusion of any one or more optional ingredient is typically at the expense of water.

With any of the described compositions and/or formulations, what is prepared may be provided in at least one form, as a ready-to-use and/or a concentrate (stock). In one or more embodiments, the concentrate will contain less water, while having a higher concentration of some or most of the components, so that a ratio of components is suitably maintained in order to obtain (by dilution) a useful and effective product when adding back water.

Described compositions and/or formulations that are prepared (as ready-to-use, as a concentrate, or by dilution of a concentrate) are useful and effective for hair and/or skin and provided to hair and/or skin to achieve at least two or more desired effects from the plurality of effects (i) to (iv) described above. Preferably, the useful and effective product once provided to hair and/or skin is left on the hair and/or skin for a period of time after application to achieve a desired effect from the two or more plurality of effects described above, which is provided after application and when at least a part of the product is removed from the hair and/or skin, in a manner utilized for a hair and/or skin product, including one or more combinations of applying on dry hair and/or skin, massaging into hair or on skin, and removing at least a part of the product by one or more of washing off, wiping off, rinsing off, combing off, and the like, or any combinations thereof. Removability (at least in part or most of the product) may be performed by any one or more of wipe-off, comb-off, brush-off, rinse-off, wash-off, and functional equivalent techniques or alternate modes understood by one of ordinary skill in the art, as is understood in the art for at least partial removing of a product described herein for use as a hair and/or skin product.

In one or more embodiments, a composition and/or formulation and/or product therefrom for hair and/or skin provides or has a cleaning and/or conditioning function with an added benefit as an anti-parasitic for hair and/or skin having or containing a parasite, or having or containing an infestation of a parasite, or suspected of having or containing a parasite, or suspected of having or containing an infestation of a parasite, the parasite being one or more of a biting and/or feeding parasite selected from one or more of a type or family of parasites including but not limited to lice, biting flies, fleas, bedbugs, mites, scabies, ticks, ectoparasites, and the like, and any combination thereof. In addition, when appropriate or as needed, any of the herein compositions and/or formulations and/or products therefrom may also be utilized to prevent infestation of or further infestation of such a biting and/or feeding parasite (one or more of a type or family of parasites including but not limited to lice, biting flies, fleas, bedbugs, mites, scabies, ticks, ectoparasites, and the like, and any combination thereof) by repeating the steps of applying on dry hair and/or skin, massaging into hair or on skin, and removing at least a part of the product by one or more of washing off, wiping off, rinsing off, combing off, and the like, or any combinations thereof. Herein compositions and/or formulations and/or products therefrom may be used prophylactically. Prophylactic and/or preventative use may include its utilization once a week, or its utilization twice a week, or its utilization every 4 days, or utilization every 5 days, or utilization every six days, or utilization every eight days, or utilization every ten days, or utilization every two weeks, or any combination thereof. Any compositions/formulations herein may become part of a normal hair and/or skin cleansing and conditioning routine.

In one or more embodiments, compositions and/or formulations herein and/or products therefrom are utilized on hair and/or skin for control of and/or removal of and/or prevention of and/or treatment against a parasite or an infestation of a parasite, the parasite being one or more of a biting and/or feeding parasite, selected from one or more of a type or family of parasites including but not limited to lice, biting flies, fleas, bedbugs, mites, scabies, ticks, ectoparasites, or any combination thereof.

In one or more embodiments, the described compositions and/or formulations and/or products therefrom are pediculicides. In one or more embodiments, the described compositions and/or formulations and/or products therefrom are pediculicides and are ovicidal (kill eggs).

Compositions and/or formulations herein do not contain one or more pyrethrins or pyrethroids. The herein described compositions and/or formulations do not contain one or more benzyl alcohols. The herein described compositions and/or formulations do not contain ivermectin. The described compositions and/or formulations do not contain malathion. The herein described compositions and/or formulations do not contain spinosad. The herein described compositions and/or formulations do not contain lindane. The herein described compositions and/or formulations do not contain alkyl sulfate(s). The herein described compositions and/or formulations do not contain silicon, silicone or silicone oils. The herein described compositions and/or formulations do not contain paraffins or paraffin oils. In some embodiments, herein described compositions and/or formulations do not contain animal wax or mineral wax or petrochemical wax or natural wax (e.g., beeswax, carnauba wax, soy wax, rice bran wax, castor wax, paraffin wax, etc.).

In some embodiments, one or more compositions and/or formulations and/or products therefrom utilized for hair and/or skin are not leave-in compositions and/or formulations (or products therefrom) for hair, nor are they leave-on compositions and/or formulations (or products therefrom) for skin. In some embodiments, one or more described compositions and/or formulations and/or products therefrom are utilized for hair and/or skin and at least in part become a leave-in composition and/or formulation (or product therefrom) for hair, or a leave-on composition and/or formulation (or product therefrom) for skin, without safety concerns, when at least some of the utilized composition and/or formulation (or product therefrom) remains in hair or on skin after being applied to hair and/or skin.

In one or more embodiments is a composition and/or formulation (or product therefrom) for hair and/or skin comprising a uniform blend, and the uniform blend includes at least the following ingredients: filtered water; a co-solvent for the filtered water; a humectant; an antimicrobial agent, wherein the antimicrobial agent may also act as a preservative; a conditioning agent; optionally a solubilizing and clarifying agent; optionally a pH balancing agent in a sufficient amount to adjust pH to between about 7.4 and greater than 5.5 (any pH therein); optionally fragrance. The uniform blend is generally provided as a spray and/or spritz and is an insect repellent and/or conditioning agent and/or detangler. Such compositions as repellants, may be left in (not removed) or combed/brushed out after remaining for a period of time. The humectant may be caprylyl glycol from 0.1-3 wt. %. Further examples are provided elsewhere herein. The antimicrobial/preservative may be at least phenoxyethanol (e.g., Argan-o-cide) from 0.5-3 wt. %. Further examples are provided elsewhere herein. The conditioning agent may be either one or both of a coconut oil derived phospholipid (e.g., Cola® Lipid C) and/or dicaprylyl carbonate from 0.5-6 wt. % (substitutions include phosphoric acid triester with N-(2,3-dihydroxypropyl)-N,N-dimethyl-3-[(1-oxococo-alkyl)amino-1-propanaminium chloride or cocamidopropyl PG-cimonium chloride phosphate or natural triglyceride, as examples). Further examples are provided elsewhere herein. When SAC agent is provided, it may be polysorbate 20 and/or decyl glucoside and/or coco glucoside and/or sodium methyl cocoyl taurate from 0-5 wt. %. Further examples are provided elsewhere herein. Fragrance when provided is often one or more essential oil, such as a proprietary blend containing lavender, rosemary leaf, lemon, eucalyptus, and lemongrass, or only one or some of the essential oils, all of which are selected for repellence properties (from 0-1 wt. %). Addition of optional components are at the expense of water.

Any of the above embodiments may be provided as a kit with a comb and/or brush.

These and other embodiments are described herein.

### DESCRIPTION OF REPRESENTATIVE EMBODIMENTS

Although making and using various embodiments are discussed in detail below, it should be appreciated that as described herein are provided many inventive concepts that may be embodied in contexts outlined and/or contemplated herein and/or interpreted or varied by the skilled artisan. Embodiments discussed herein are merely representative and do not limit the scope of the invention.

In some embodiments, a composition and/or formulation described herein is prepared and provided as a personal care product for hair and/or skin and is at least a cleansing product. As a cleansing product, it is sufficient to clean hair and/or skin by performing at least one or more of removal of unwanted sebum, removal of dandruff, removal of environmental dirt and dust, and removal of residues or build-up on hair and/or skin after its application in a manner described herein. The cleansing product may be in a form of a shampoo and/or mousse and/or cream and/or balm and/or paste and/or spray.

In some embodiments, a composition and/or formulation described herein is prepared and provided as a personal care product for hair and/or skin and is at least a conditioning product. As a conditioning product, it is sufficient to condition hair and/or skin by leaving conditioning components onto hair and/or skin for one or more of protection, moisturizing, removing tangles, increasing elasticity and increasing shine of hair and/or skin. The conditioning product may be in a form of a shampoo and/or mousse and/or cream and/or balm and/or paste and/or spray and/or spritz.

In one or more embodiments, a composition and/or formulation described herein is prepared and provided as a personal care product for hair and/or skin and is at least a product having anti-parasitic activity. As an anti-parasitic product, it has activity against parasites, killing and/or knocking down one or more of lice, biting flies, fleas, bedbugs, mites, scabies, ticks, and any combination thereof, after application to the hair and/or skin for a period of time followed by removal (at least in part) from the hair and/or skin. In one or more forms, the anti-parasitic product may be in a form of a shampoo and/or mousse and/or cream and/or paste and/or balm and/or spray.

In one or more embodiments, a composition and/or formulation described herein is prepared and provided as a personal care product for hair and/or skin and is at least a product having prophylactic activity against a parasite. As a prophylactic product, it is a sufficient prophylaxis for prevention of further infestation of one or more of lice, biting flies, fleas, bedbugs, mites, scabies, ticks, or other biting or feeding parasite described, and any combination thereof. Prophylaxis and or prevention is obtained after at least one repeated application to the hair and/or skin for a period of time followed by removal (at least in part) from the hair and/or skin. In one or more forms, the prophylactic or preventative product is provided as a shampoo and/or mousse and/or cream and/or balm and/or paste and/or spray.

Compositions and/or formulations herein contain a plurality of ingredients, which are carefully composed and formulated and in amounts described herein in order to impart a product with a multi-functional effect, selected from at least two of: (i) anti-parasitic (having a sufficient killing and/or knock down effect against one or more parasites being at least one or more of lice, biting flies, fleas, bedbugs, mites and ticks in order to achieve sufficient removal of the one or more parasites from the hair and/or skin); (ii) prophylactic and/or preventative (having sufficient prophylactic or preventative effect in order to prevent re-infestation on hair and/or skin of one or more parasites being at least one or more of lice, biting flies, fleas, bedbugs, mites and ticks); (iii) cleansing (having sufficient cleaning effect to clean hair and/or skin by performing at least one or more of removal of unwanted sebum, removal of dandruff, removal of environmental dirt and dust, and removal of residues from hair and/or skin); and (iv) conditioning (having sufficient conditioning effect to condition hair and/or skin by leaving conditioning components onto hair and/or skin for one or more functions, the functions being any one or more of protection, moisturizing, removing tangles, increase elasticity and increasing shine). Products composed and/or formulated as described and in the described amounts may impart a multi-functional effect that includes four functions, anti-parasitic, prophylactic or preventative, cleansing, and conditioning.

Unique to the compositions and/or formulations described herein is having an alkyl polyglucoside (APG) as an active ingredient (insecticide) and as primary surfactant, which is a nonobvious and unexpected manner of utilizing APG, in a nonobvious and unexpected quantity. Further components in compositions and/or formulations herein are found necessary to ensure performance when provided as a final product, including for uniform blending and mixing, overall homogeneity, stability, clarity, suppression of splitting, handleability, and shelf-life, so that the product, and in particular the APG, being in such an unusual high quantity as an active ingredient and primary surfactant, will remain stable, will remain blended and suspended (clarified), and does not aggregate (e.g., floc or precipitate) or split.

The nonobvious and unexpected utilization described herein of an APG as an active ingredient in an insecticide and as a primary surfactant in that insecticide, in which the insecticide has shampoo and conditioning properties for hair and/or skin care and/or preventative properties against re-infection of an insect described herein, is contrary to common knowledge and to present use of an APG. In fact, alternative hair or skin products, which are hair or skin cleansing products are not anti-parasitic or prophylactic against insect re-infestation, nor do they have APG as a primary surfactant in such a high quantity. In so-called alternative hair or skin products, APG is provided as an additive (inactive and non-anti-parasitic component) and in a low quantity, typically 9% or less, or 8% or less, or 6% or less, or 5% or less, or less than 5%. In such so-called alternatives, an APG is not an active ingredient for anti-parasitic activity, meaning said so-called alternatives are not truly alternatives because they are not anti-parasitic and the APG in such non-anti-parasitic alternatives is not provided for utility as an anti-parasitic agent for killing and/or knock-down of one or more parasites (e.g., lice, biting flies, fleas, bedbugs, mites, scabies, ticks, and combinations thereof). It is not obvious to utilize APG for purposes described herein (e.g., to provide multiple functional effects (i) to (iv) as described above). And, it is contrary to a typical utilization of APG in a ready-to-use personal care product for hair and/or skin to have APG as a primary surfactant or to have APG in higher amounts than typically utilized in alternative products for hair or skin.

A quantity of one or more APG provided in a composition and/or formulation herein is often much more than 10% of the total composition. In one or more embodiments, APG in total in a composition and/or formulation herein is at or about or more than about 13% of the total composition, or is at or about or more than about 14% of the total composition, or is at or about or more than about 15% of the total composition, or is at or about or more than about 20% of the total composition, or is at or about or more than about 30% of the total composition, or is greater than about 30% of the total composition. In some embodiments, APG in a composition and/or formulation herein is at least about 25% higher, or more than about 25% higher than an amount of APG found in a typical and so-called alternative personal care product for hair or skin. In some embodiments, APG is at least about 30% higher, or more than about 30% higher than an amount of APG found in a typical and so-called alternative personal care product for hair or skin. In some embodiments, APG amount in total is at least twice as high, or more than twice as high as an amount of APG found in a typical and so-called alternative personal care product for hair or skin.

As is known in the relevant field, an APG is typically available (e.g., manufactured in a manufacturing process by a chemical manufacturer) as a concentrate, which is a semi-solid or very viscous liquid with a high solids content. Generally, in such a raw form, which is a high solids form, a solids content is at or greater than about 50% (or, sometimes, from about 46% to about 55% or up to about 60% or up to about 65% or up to about 70%). In addition, in such a raw form, or a high solids form, APG is generally at a pH at about or greater than about 11, or between about pH 11 and about pH 12, or between about pH 11.5 and about pH 12.5, or between about pH 11 and about pH 13.

Because typical hair or skin products that are ready-to-use, have a much lower pH (neutral or acidic), this poses a difficulty because when pH is lowered, even to a more neutral pH, such as between pH 7 or pH 8, because an APG as a concentrate, or at a high solids concentration (e.g., greater than 13%, or at or up to about or greater than about 15%, or at or up to about or greater than about 20%, or at or up to about or greater than about 25%, or at or up to about or greater than about 30%, or at or up to about or greater than about 35%, or at or up to about or greater than about 40%, or up to about or greater than about 45%, or in any range there between) will naturally lose stability (becomes less stable), becomes less soluble, and will tend to separate (e.g., split) and/or aggregate (e.g., floc or precipitate). It is generally understood by those of ordinary skill in the art that having APG at such a high concentration (as utilized herein) and at a lowered pH (e.g., pH<8, pH near neutral pH, about neutral pH, or acidic pH), the APG becomes insoluble, can form precipitates that settle (as dense white particles), and a product therefrom will either split or thicken (e.g., cream or stick) to a point that makes the product unmanageable (splitting, thickening and/or stickiness prevent flow and movement, and prevent adequate saturation of a surface, such as hair or skin). It is also difficult to prolong shelf-life of APG at a higher concentration.

In view of the above, it is not obvious or expected to utilize APG at high concentrations (amounts) described herein. And, utilizing APG in quantities described herein as an active component and as a primary surfactant, as well as adjusting its pH in order to utilize it as a hair and/or skin product, required a number of additional and unexpected combination of components to be included for utility and stability, in which said combination of additional components is not obvious or anticipated, requiring numerous formulary adjustments in order to arrive at utilitarian and stable compositions and/or formulations (and products therefrom) that displayed the herein desired characteristics and effects. The plurality of additional components (ingredients) included with APG to arrive at one or more inventive compositions and/or formulation described herein (that are suitable to be utilized for hair and/or skin as described) had to, in combination, provide desired characteristics, such as proper effectiveness (anti-parasitic property), good stability, uniform blending, clarification, and extended shelf-life (months to years), and without any separation or splitting or aggregation (e.g., no floc formation or precipitation) of a composition and/or formulation, or product therefrom. The one or more inventive compositions and/or formulation described herein do not include another primary non-ionic surfactant, which is typically found in a so-called alternative product for hair or skin. In one or more embodiments, the one or more compositions and/or formulations herein do not include any of disodium laureth sulfosuccinate, sodium coco sulfate, sodium lauryl sulfate, sodium lauryl ether sulfate, or cocoimidopropyl betaine.

One or more APG as described herein is utilized in any of said one or more inventive compositions and/or formulations as a primary surfactant (and insecticide). The amount in a final composition and/or formulation or product therefrom (ready-to-use), based on weight of the final composition and/or formulation or product therefrom, may be greater than about 13%, or greater than about 14%, or greater than about 15%, or greater than about 16%, or greater than about 17%, or greater than about 18%, or greater than about 19%, or greater than about 20%, or greater than about 21%, or greater than about 22%, or greater than about 23%, or greater than about 24%, or greater than about 25%, or greater than about 26%, or greater than about 27%, or greater than about 28%, or greater than about 29%, or greater than about 30%, or greater than about 31%, or greater than about 32%, or greater than about 33%, or greater than about 34%, or greater than about 35%, or greater than about 36%, or greater than about 37%, or greater than about 38%, or greater than about 39%, or greater than about 40%, or greater than 10% and up to about 45%, or greater than 15% and up to about 45%, or greater than about 15% and up to about 40%, or at or greater than about 15% and up to about 35%, or greater than 20% and up to about 45%, or greater than 20% and up to about 40%, or greater than 20% and up to about 35%, or greater than 25% and up to about 45%, or greater than 25% and up to about 35%. In addition or as an alternative, an APG utilized in any of said one or more inventive compositions and/or formulations as a primary surfactant, in a concentrate, is in an amount that is: between about 45% and about 70%, between about 45% and about 65%, or between about 45% and about 60%, or between about 45% and about 55%, or between about 45% and about 70%, or between about 50% and about 70%, or between about 50% and about 65%, or between about 50% and about 60%, or between 50% and about 55%, or between about 50% and about 75%, or based on weight of a total composition.

An APG for compositions and/or formulations herein is biodegradable and plant-derived, having a hydrophilic head group (made from a naturally sourced glucose) and a hydrophobic carbon-chain tail (derived from a naturally sourced oil converted to fatty alcohols). When processed, an APG will generally contain a mixture of isomers as well as a range of tail lengths. For example, lauryl glucoside may be C12-C14, decyl glucoside may be about 60% C8-C10 and about 40% C12-C14 (or C8-C10>C12-C14 alkyl chains), and coco glucoside may be about 40% C8-C10 and about 60% C12-C14 (or C8-C10<C12-C14 alkyl chains). A further purification step may be taken (to control purity, including carbon chain length (alkyl chain)) if desired, or to include distinct fatty alcohol carbon chain length(s). Herein, when APG is identified by chain length, it is an average (e.g., median) chain length since many fatty alcohols are supplied by natural and/or renewable feed stocks, so that the designated length is the average (e.g., median) length. In some but not all embodiments, an APG is selected as one that is minimally processed.

In typical (so-called alternative) personal care products for skin or hair, in which, in a ready-to-use form (e.g., wipe-off, brush-off, rinse-off, comb-off, and/or wash-off and the like), and are not considered, on their whole, as long-term stay-on products, when an APG is used, the amount is low because each APG is highly viscous, also prone to thermal degradation and oxidation. Thus, when used in so-called alternative products, APG is always in a very low amount (often less than 5% of a total composition). So, while such a so-called alternative product (e.g., as an alternative shampoo or mousse) may contain an APG (e.g., decyl glucoside, arachidyl glucoside, C12-20 alkyl glucoside, caprylyl/capryl glucoside, cetearyl glucoside, coco-glucoside, ethyl glucoside), when the APG is one that contains a longer chain fatty alcohol (e.g., from C12-C20, or is a laurylic glucoside) it is not utilized as the primary surfactant in the alternative product, and, instead, is a secondary surfactant, added to alleviate harshness of many primary surfactants (ones not an APG) that are non-ionic, or added as a foaming agent because it is known that glucosides having longer chain alkyl groups (e.g., from C12-C20) will provide more foam.

For compositions and/or formulations herein, an APG may be exemplified by, but is not limited to, at least one of or more than one of: decyl glucoside, arachidyl glucoside, butyl glucoside, C10-C16 alkyl glucoside, C12-C18 alkyl glucoside, C12-C14 alkyl glucoside, C12-C16 alkyl glucoside, C12-C18 alkyl glucoside, C12-20 alkyl glucoside, C10-C16 alkyl glucoside, C20-22 alkyl glucoside, ascorbyl glucoside, caprylyl/capryl glucoside, caprylyl glucoside, cetearyl glucoside, coco-glucoside, ethyl glucoside, octyl glucoside, hexadecyl D-glucoside, isostearyl glucoside, lauryl glucoside, myristyl glucoside, dodecyl D-glucoside, octadecyl D-glucoside, octyldodecyl glucoside, undecyl glucoside, and any combination thereof, in which a lauryl glucoside may include C12-C14, decyl glucoside may include about 60% C8-C10 and about 40% C12-C14 (or C8-C10>C12-C14), and coco glucoside may include about 40% C8-C10 and about 60% C12-C14 (or C8-C10<C12-C14).

In some preferred embodiments, for compositions and/or formulations herein, an APG utilized herein is one having an alkyl (carbon) chain length that is, on average, or in predominance (e.g., greater than 50%, or at or greater than 55%, or at or greater than 60%, the percentages based on all alkyl group (carbon atom) chain lengths) at least 12 carbon atoms or greater, or is a combination of on average or in predominance any one or more of 10 and 12 carbon atoms, and/or is a combination of on average or in predominance any one or more of 10 and 12 and 14 carbon atoms, and/or is a combination of on average or in predominance any one or more of 12 and 14 carbon atoms, or some combination thereof. For example, in one or more embodiments, and in some preferred embodiments, for compositions and formulations herein, the APG utilized herein is or is predominantly a lauryl glucoside or is predominantly a C12-C14 alkyl glucoside. In some preferred embodiments, for compositions and/or formulations herein, the APG utilized herein is or is predominantly a coco glucoside or about 40% C8-C10 and about 60% C12-C14 or C8-C10<C12-C14. In some preferred embodiments, for compositions and/or formulations herein, the APG utilized herein is or is predominantly a decyl glucoside or about 60% C8-C10 and about 40% C12-C14 or C8-C10>C12-C14. In some preferred embodiments, the utilized herein APG is or is predominantly a C8-C14 alkyl glucoside. In some preferred embodiments, the utilized herein APG is or is predominantly a C10-C14 alkyl glucoside. In some preferred embodiments, the utilized herein APG is or is predominantly a C10-C16 alkyl glucoside. In some preferred embodiments, the APG utilized herein is or is predominantly a C12-C16 alkyl glucoside. In some preferred embodiments, the APG utilized herein is or is predominantly a C8-C16 alkyl glucoside. In some preferred embodiments, the APG utilized herein is selected from any one or more of lauryl glucoside; C12-C14 alkyl glucoside; decyl glucoside (in quantity or concentration: C8-C10 alkyl chains>C12-C14 alkyl chains); coco glucoside (in quantity or concentration: C8-C10 alkyl chains<C12-C14 alkyl chains); dodecyl D-glucoside; C12-C16 alkyl glucoside; C12-C18 alkyl glucoside; C10-C16 alkyl glucoside; and/or having any one or more of C8-C20 alkyl chains and predominantly any one or more of a C8 to C14 alkyl chains; and any combination thereof. In some preferred embodiments, the APG utilized herein is any one or more of a lauryl glucoside; a decyl glucoside, a coco glucoside, a C10-C16 alkyl glucoside; a C12-C14 alkyl glucoside; a D-glucopyranose, oligomeric, C10-C16-alkyl glycoside; a glucopyranose, oligomeric, C10-C16-alkyl glycoside. In some preferred embodiments, the APG utilized herein is utilized alone. In some preferred embodiments, APG utilized herein is utilized in a combination of at least two APGs being in predominance from two of: C12-C14 alkyl glucoside; decyl glucoside (in predominant quantity or concentration: C8-C10 alkyl chains>C12-C14 alkyl chains); coco glucoside (in predominant quantity or concentration: C8-C10 alkyl chains<C12-C14 alkyl chains); lauryl glucoside (in predominant quantity or concentration: C12-C14 alkyl chains); dodecyl D-glucoside; C12-C16 alkyl glucoside; C10-16 alkyl glucoside; C8-C16 alkyl glucoside; C10-C16 alkyl glucoside; and/or having any one or more of C6-20 alkyl chains and predominantly any one or more of a C8 to C14 alkyl chains; and any combination thereof. It is noted that an APG may be referred to by differing chemical names; lauryl glucoside is a scientific or INCI name (International Nomenclature of Cosmetic Ingredients), and may also be referred to as: dodecyl glucoside, dodecyl b-D-glucopyranoside, and dodecyl beta-D-glucopyranoside.

APGs having a carbon chain length of C6 or less are generally not selected and are not predominant for preparations herein (although, in an APG batch or in an APG preparation, there may be a quantity having a carbon chain length of C6 or less, but said quantity will be less than 25%, or less than 20%, or less than 15%, or less than 10%, or less than 5%, or less than 1%, based on the total APG in said APG batch or APG preparation, or some range thereof). APGs having a carbon chain length of C18 or greater are generally not selected and are not predominant in APG preparations utilized herein (although, in an APG batch or in an APG preparation, there may be a quantity having a carbon chain length of C18 or greater, in which said quantity will generally be less than 25%, or less than 20%, or less than 15%, or less than 10%, or less than 5%, or less than 1% of the total APG in said APG batch or APG preparation).

In many embodiments, for compositions and/or formulations herein, an APG stock utilized for a preparation herein is one that is about 50%-55% active (or about 45%-60% APG in an aqueous solution, pH>11). While an APG stock may include alkyl (carbon atom) chain lengths in a range from between 6 to 20 carbons, in preferred embodiments, a predominant alkyl (carbon) chain length in an APG will be 10 and greater, or 12 and greater, or 8 and greater (e.g., predominantly C10-C14 alkyl chain; predominance being greater than at least 50%, or at or greater than 55%, or at or greater than 60%).

In some embodiments, for compositions and/or formulations herein, an APG is selected as one that has a low skin irritation index. It is understood that the skin irritation index declines the higher the alkyl (carbon) chain length is. An APG having a lower skin irritation index is considered safer.

In some preferred embodiments, for compositions and/or formulations herein, an APG may be one that is a lauryl glucoside, utilized alone. It is noted that while an APG such as lauryl glucoside is in some so-called alternative skin or hair products for foaming or shampoo applications (because it has a good safety profile, a low irritation index and is considered mild and suitable for all skin or hair types, including babies or young children and/or sensitive or irritated skin), the lauryl glucoside (nor any APG or combination herein) is not provided as a primary surfactant in the so-called alternatives, nor is it provided in quantities described herein because its pH is too high, while skin or hair product requires a lower pH that is neutral or acidic. The lauryl glucoside (or any APG or combination herein) would be unsuitable on its own (as an only surfactant), being unstable and significantly less soluble at the neutral or acidic pH.

In one or more embodiments, and in some preferred embodiments, for compositions and/or formulations herein, an APG may be one that is a lauryl glucoside utilized in combination with any one or more APGs described above or as claimed (e.g., coco glucoside or about 40% C8-C10 and about 60% C12-C14 or C8-C10<C12-C14; decyl glucoside or about 60% C8-C10 and about 40% C12-C14 or C8-C10>C12-C14; predominantly C8-C14 alkyl glucoside; predominantly C12-C16 alkyl glucoside; predominantly a C8-C16 alkyl glucoside; predominantly C10-C16 alkyl glucoside; predominantly C10-C14 alkyl glucoside; predominantly C10-C16 alkyl glucoside; decyl glucoside (predominantly in quantity or concentration: C8-C10 alkyl chains>C12-C14 alkyl chains); coco glucoside (predominantly in quantity or concentration: C8-C10 alkyl chains<C12-C14 alkyl chains); and/or having any one or more of C6-C20 alkyl chains and predominantly any one or more of a C8 to C14 alkyl chains; and any combination thereof).

Herein compositions and/or formulations (as an insecticide (i), prophylaxis against re-infestation (ii), cleanser (iii), and/or conditioner (iv)) are water-based, and will contain water and a co-solvent or carrier for the water in addition to APG. Three or more from a plurality of additional ingredients (components, identified below) are also included in each composition and/or formulation, which may be ready-to-use or in a concentrate. For a concentrate, an amount of water will decrease as the amount of each non-water component is increased. It is understood that in a concentrate, a proportion of non-water components will, with respect to each other, remain substantially the same, and when properly diluted with water (as described and exemplified herein), will then provide a suitable ready-to-use composition and/or formulation. It is noted that both a ready-to-use or a concentrate may be utilized and are effective for purposes herein (e.g., having two or more of effects (i) to (iv)), while ready-to-use compositions and/or formulation (and products therefrom) will provide more cost savings during manufacturing.

The plurality of additional ingredients (components) in compositions and/or formulations herein, whether ready-to-use or concentrate, are selected from at least three or more of the following: pH balancer, antimicrobial/preservative, humectant, chelating agent, solubilizing and clarifying agent, and conditioning agent. Ready-to-use compositions/formulations or products therefrom when in a final blend (containing APG, water, co-solvant or carrier for water, and three or more of pH balancer, antimicrobial/preservative, humectant, chelating agent, solubilizing and clarifying agent, and conditioning agent, without further additives) are clear solutions and, having a viscosity similar to an oil (more viscous than water). Without further additives (e.g., essential oils or fragrance), a composition/formulation herein has little or no scent or is odorless.

A conditioning agent and/or a solubilizing and clarifying (SAC) agent is/are optional. In some embodiments, a herein composition and/or formulation will comprise at least one SAC agent for stability and uniformity of the blend comprising one or more APG. In some embodiments, a herein composition and/or formulation will comprise at least two SAC agents for stability and uniformity of the blend comprising one or more APG. In some embodiments, a herein described novel composition and/or formulation for hair and/or skin will include three or more of the plurality of additional ingredients. In some embodiments, a herein described novel composition and/or formulation for hair and/or skin will include four or more of the plurality of additional ingredients. In some embodiments, a herein novel composition and/or formulation for hair and/or skin will include five or more of the plurality of additional ingredients. In some embodiments, a conditioning agent is not required since a combination of at least the one or more APG, with at least the one or more solubilizing and clarifying agents, together provide a sufficient conditioning effect to condition hair and/or skin by leaving conditioning components onto hair and/or skin for one or more of protection, moisturizing, removing tangles, increase elasticity and increasing shine. It is contemplated and not beyond what is understood by one skilled in the art, that in one or more embodiments herein, a further additive may be included in a multi-functional composition and/or formulation or product therefrom as described, without compromising amounts of certain critical ingredients (APG, co-solvent, and three or more of a plurality of additional ingredients, selected from pH balancer, antimicrobial/preservative, humectant, chelating agent, solubilizing and clarifying agent, and conditioning agent). A further additive may be a thickener (such as emollient, natural fatty acid, natural butter, etc.) to create a thicker product, such as a paste, balm, or cream, as is understood in the relevant field of hair and/or skin products. In addition or as an alternative, a further additive may be an essential oil or fragrance having some insect repellence. In addition or as an alternative, a further additive may be a hair or skincare enzyme. In addition or as an alternative, a further additive may be a natural colorant or pigment. Any one or more of the further additive, when included, will be one that is safe and permitted for use in a skin and/or hair composition and/or formulation and for a personal care product for skin and/or hair. In some embodiments, there is no fragrance (no essential oils) and no colorant in a useful and multi-functional composition and/or formulation. In some embodiments, there is no hair or skincare enzyme in a useful and multi-functional composition and/or formulation. In some embodiments, there is no added thickener in a useful and multi-functional composition and/or formulation.

Generally, none of the ingredients or components are animal ingredients or components. In one or more embodiments, all the ingredients or components are considered cruelty free.

Generally, in one or more embodiments, no pesticide is included as an ingredient or a component in a composition and/or formulation described herein.

In one or more embodiments, most of the ingredients or components are from or derived from a natural source. In one or more embodiments, all the ingredients or components are from or derived from a natural source. In one or more embodiments, at least one, or at least some of the ingredients or components described herein are renewable. In one or more embodiments, all the ingredients or components described herein and provided in a composition and/or formulation described herein are renewable. In one or more embodiments, at least one, or at least some of the ingredients or components described herein are biodegradable. In one or more embodiments, all the ingredients or components described herein and provided in a composition and/or formulation described herein are biodegradable. In one or more embodiments, most of the ingredients or components described herein are natural, renewable, and biodegradable. In one or more embodiments, all the ingredients or components described herein and provided in a composition and/or formulation described herein or in a product therefrom, for hair and/or skin, are natural, renewable, and biodegradable.

In one or more embodiments, at least some of the ingredients or components described herein are natural and organic. In one or more embodiments, all the components described herein and provided in a composition and/or formulation described herein or product therefrom, for hair and/or skin, are natural and organic, certified by any one or more certifying agencies in the U.S. or outside the U.S. (when utilized outside the U.S.). Representative examples include: COSMOS organic (COSMOS being COSMetic Organic and natural Standard, certified by ECOCERT); ANSI 305 (ANSI being American National Standards Institute established by NSF International); the National Organic Program (established by the US Department of Agriculture); OASIS (OASIS being Organic and Sustainable Industry Standards, established by the cosmetic industry). Additional certification and/or guidelines for personal care products and compositions and/or formulations for skin and/or hair described herein may be provided by any one or more organizations, exemplified by: Natural Products Association (e.g., Natural Standard for Personal Care Products, and NPA Natural Seal); Certech (e.g., IOS Cosmetic Standard); California Department of Public Health (e.g., California Safe Cosmetics Program, California Safe Cosmetics Act); Personal Care Products Council, European Union (e.g., EU Council Directive76/768/EEC). One or more or all herein compositions and/or formulations or products therefrom are or may be certified by one or more organizations and/or current standards establishing good and safe ingredients, as well as for quality and/or safety standards for personal care products for skin and hair.

In one or more embodiments, a suitable composition and/or formulation as an insecticide will comprise all the following components: at least one APG, water, at least one co-solvent or carrier for water, at least one pH balancer, at least one humectant, at least one chelating agent, and at least one antimicrobial/preservative. More than one of some components may be included in some compositions and/or formulations for hair and/or skin.

In one or more embodiments, a suitable composition and/or formulation as an insecticide will comprise all the following components: at least one APG, water, at least one co-solvent or carrier for water, at least one pH balancer, at least one humectant, at least one chelating agent, at least one antimicrobial/preservative, and at least one conditioning agent. More than one of some components may also be included in some compositions and/or formulations for hair and/or skin.

In some embodiments, a suitable composition and/or formulation as an insecticide will comprise all the following components: at least one APG, water, at least one co-solvent or carrier for water, at least one pH balancer, at least one humectant, at least one chelating agent, at least one antimicrobial/preservative, and at least one solubilizing and clarifying agent. More than one of some components may also be included in some compositions and/or formulations for hair and/or skin.

In one or more embodiments, a suitable composition and/or formulation as an insecticide will comprise all the following components: at least one APG, water, at least one co-solvent or carrier for water, at least one pH balancer, at least one humectant, at least one chelating agent, at least one antimicrobial/preservative, water, at least one solubilizing and clarifying agent, and at least one conditioning agent. More than one of some components may also be included in some compositions and/or formulations for hair and/or skin.

In some embodiments, a suitable composition and/or formulation as an insect repellant will comprise at least all the following components: water, at least one co-solvent or carrier for water, at least one humectant, at least one antimicrobial/preservative, at least one conditioning agent, with or without fragrance. More than one of some components may be included.

In some embodiments, a suitable composition and/or formulation as an insect repellant will comprise at least all the following components: water, at least one co-solvent or carrier for water, at least one humectant, at least one antimicrobial/preservative, at least one conditioning agent, and at least one SAC agent, with or without fragrance. More than one of some components may be included.

In any of the herein described embodiments, one or more further additive may be included, when desired, without compromising integrity of one or more effects (i) to (iv) described elsewhere, in which the one or more further additive may comprise any one or more of: fragrance, enzymes, pigments/colorant, and/or thickener.

Any composition and/or formulation described herein, with or without at least one further ingredient or component, may be provided in a ready-to-use topical form, or in a topical concentrate.

Compositions and/or formulations herein, whether in a ready-to-use form or as a concentrate, may be provided in final form as a spray, spritz, shampoo, mousse, and the like, which are suitable forms for use on hair and/or skin, as well as any one or more of a cream, gel, paste, balm (when a thickener is added). In some embodiments, a composition and/or formulation will be provided as a removable composition and/or formulation, utilized as a removable hair or skin composition and/or formulation or product therefrom (removing some or all from hair or skin).

In view of the above, and for purposes of understanding the description herein, it is generally provided that inventive compositions and/or formulations are applied to hair and/or skin, remain on hair and/or skin for a period of time, and then removed at least in part from the hair and/or skin. An acceptable period of time to allow a composition or formulation to remain may be up to ten minutes, or up to fifteen minutes, or up to about thirty minutes, or up to 45 minutes, or up to about an hour, or for a couple hours. Thirty minutes or longer will provide better short term killing and/or knock down of one or more parasites. In general, an inventive composition and/or formulation can remain for up to a couple hours on hair without hair and/or skin irritation, and may also be re-applied a same day or the next day or for several days. Importantly, because the inventive composition and/or formulation is not an irritant to the hair and/or skin, it can replace shampoo and be used regularly, which provides effective prophylaxis to reduce and/or eliminate re-infestation of one or more parasites (data not shown). The composition and/or formulation is not required to remain on hair and/or skin for an extended period (e.g., more than a couple hours, all day, or overnight). Exemplary embodiments are provided in the accompanying tables and are effective compositions and/or formulations having anti-parasitic activity.

A composition and/or formulation herein may be in one of a number of forms, such as shampoo, mousse, spray, spritz, when prepared (without thickener), and will include water, making up more than one-third (33%) of a ready-to-use composition and/or formulation. Water may be purified water. Water may be a distilled water (with little or no contaminants or minerals). Water may be a deionized water. Water may be filtered water. Water may also be filtered and purified water. The amount of water in a composition and/or formulation described herein will generally be from about 20 wt. % to about 70 wt. % based on the total weight of the composition and/or formulation, or in any range there between. In some embodiments, the amount of water will be from about 20 wt. % to about 50 wt. %. In some embodiments, the amount of water will be from about 25 wt. % to about 45 wt. %. In some embodiments, the amount of water will be from about 40 wt. % to about 60 wt. %. In some embodiments, the amount of water will be from about 20 wt. % to about 60 wt. %. In some embodiments, the amount of water will be from about 45 wt. % to about 70 wt. %. In some embodiments, the amount of water will be up to or at about 55 wt. %. The amount is based on the total weight of the composition and/or formulation, or may be in any range there between. In certain concentrated embodiments, the amount of water may be at about or less than about 20 wt. %.

A co-solvent or carrier for water (H₂O) is or may be derived from at least one of a group provided herein, or may be a functional and/or chemical equivalent therein, functionally active and/or chemically active derivatives thereof, and/or functionally active and/or chemically active analogs thereof, understood by one of skill in the art as acting as a co-solvent or carrier for water for a skin and/or hair composition and/or formulation herein. Representative, and non-limiting examples of a co-solvent include, but are not limited to, organic agents (e.g., molecules or chemicals) that aid in dissolving, or dispersing or liquefying or solubilizing a solid liquid or gaseous solute (as well as fats or oils) of the composition and/or formulation. Such a co-solvent for hair or skin products commonly include a low boiling point alcohol or alcohol-containing chemical or acetate ester. Illustrative and further representative examples of a suitable co-solvent for compositions and/or formulations described herein include, but are not limited to: glycol, polyethylene glycol, glycol ether, glycol ether esters, butylene glycol, propanediol, glycerol, ethoxydiglycol, alkyl benzoate, sodium benzoate, aloe vera palmitate, ethylhexyl palmitate, ethyl lactate (and esters of lactic acid), isolanolin, 2-butoxyethanol (butyl), dehydrated alcohol, isopropyl alcohol (rubbing alcohol), and d-limonene. In one or more embodiments, a safe co-solvent or carrier is utilized. In one or more embodiments, a safe co-solvent or carrier is one that contains exceptionally low level or no level of hazardous air pollutants (HAPs) emission, has low or no volatile organic compounds (VOCs), and/or one with little, low or no flashpoint (generally having little or low flammability), or is inflammable or considered non-flammable. In one or more embodiments, a co-solvent or carrier is a glycol or a type of glycol or a modified glycol. An example includes, but is not limited to polyethylene glycol. In one or more embodiments, a co-solvent or carrier is a propanediol, or a functional equivalent, or a structural analog or derivative having a same or similar function. The amount of a co-solvent in a composition and/or formulation described herein that is a ready-to-use composition and/or formulation will be from about 1 wt. % to about 20 wt. %. In some embodiments, the co-solvent will be in an amount from about 1 wt. % to about 15 wt. %. In some embodiments, the amount of the co-solvent will be from about 5 wt. % to about 15 wt. %. In some embodiments, the amount of the co-solvent will be from about 8 wt. % to about 15 wt. %. In some embodiments, the amount of the co-solvent will be up to about 15 wt. %. In some embodiments, the amount of the co-solvent will be up to about 14 wt. %. In some embodiments, the amount of the co-solvent will be up to about 13 wt. %. In some embodiments, the amount of the co-solvent will be up to about 12 wt. %. The amount is based on a total weight of the composition and/or formulation, and/or may be in any range there between.

A pH balancer herein is or may be derived from at least one of a group provided herein, or may be a functional and/or chemical equivalent therein, functionally active and/or chemically active derivatives thereof, and/or functionally active and/or chemically active analogs thereof, understood by one of skill in the art as acting as a pH balancer for a hair and/or skin composition and/or formulation herein. Representative, and non-limiting examples of a pH balancer for compositions and/or formulations described herein include, but are not limited to, substances (e.g., solutions, molecules or compounds) that assist in arriving at a particular pH or pH range and/or maintain a particular pH or pH range of the composition and/or formulation described herein. Illustrative and further representative examples of a suitable pH balancer for compositions and/or formulations described herein include, but are not limited to: mild acids, such as citric acid, salicylic acid, lactic acid, as representative examples. The pH balancer will be utilized to reduce the pH, because without a pH balancer, pH of a composition and/or formulation is alkaline (due to a high amount of the at least one APG). In one or more embodiments, a pH balancer will be utilized to adjust pH of the composition and/or formulation to less than pH 8.0. In one or more embodiments, a pH balancer will be utilized to adjust pH of the composition and/or formulation so that pH is between about 7.4 and greater than about 6.0. In one or more embodiments, a pH balancer will be utilized to adjust pH of the composition and/or formulation so that pH is between about 7.4 and about 6.4. In one or more embodiments, a pH balancer will be utilized to adjust pH of the composition and/or formulation so that it is at or about pH 6.5, or about pH 6.6, or about 6.7, or about 6.8, or about 6.9, or about 7, in an amount as needed or Q.S. The amount of a pH balancer in a composition and/or formulation described herein will often be from about 0.05 wt. % to about 5 wt. %. In some embodiments, the amount of the pH balancer will be from about 0.1 wt. % to about 4 wt. %, or up to about or at about 3 wt. %, or up to about or at about 2 wt. % (the amount being based on a total weight of the composition and/or formulation, and in any range there between).

An antimicrobial/preservative herein is or may be derived from at least one of a group provided herein, or may be a functional and/or chemical equivalent therein, functionally active and/or chemically active derivatives thereof, and/or functionally active and/or chemically active analogs thereof, understood by one of skill in the art as acting as an antimicrobial/preservative for a skin and/or hair composition and/or formulation herein. Representative, and non-limiting examples of an antimicrobial/preservative for compositions and/or formulations described herein include, but are not limited to, substances (e.g., molecules or compounds) that protect the compositions and/or formulations described herein against the natural effects of aging such as decay, discoloration, oxidation and/or bacterial degradation, and increase shelf-life. Illustrative and further representative examples of an antimicrobial/preservative for compositions and/or formulations described herein include, but are not limited to: glycol ethers, caprylyl glycol, isothiazolinones, citricidals, isopentyldiol, phenoxyethanol, formaldehyde releaser, urea-containing substances, hydantoin-containing substances, stannic chloride, EDTA, organic acid (e.g., benzoic acid/sodium benzoate, sorbic acid/potassium sorbate, levulinic acid, anisic acid), benzalkonium chloride, parabens, triclosan, ethyl lauroyl arginate. More than one antimicrobial/preservative may be used in a composition and/or formulation described herein. In one or more embodiments, more than one antimicrobial/preservative is preferred to ensure good shelf-life of a product containing a large quantity of APG, as described herein. In one or more embodiments, the antimicrobial/preservative is an organic chemical. In one or more embodiments, the antimicrobial/preservative is at least a phenoxyethanol. In one or more embodiments, the antimicrobial/preservative is at least a caprylyl glycol. In one or more embodiments, the antimicrobial/preservative is not a paraben, and does not contain a paraben. The amount of an antimicrobial/preservative in a composition and/or formulation described herein will be from about 0.05 wt. % to about 4 wt. %. In some embodiments, the amount will be from about 0.05 wt. % to about 3 wt. %. In some embodiments, the amount will be from about 0.1 wt. % to about 2 wt. %. In some embodiments, the amount will be from about 0.1 wt. % to about 1 wt. %. In some embodiments, the amount will be up to about or at about 4 wt. %. In some embodiments, the amount will be up to about or at about 3 wt. %. In some embodiments, the amount will be up to about or at about 2 wt. %. In some embodiments, the amount will be up to about or at about 1 wt. %. The amount is based on a total weight of the composition and/or formulation, and/or may be in any range there between.

It is noted that in some embodiments, at least one ingredient may be multi-functional (e.g., behave or function for more than one purpose). Some chelating agents may be multi-functional by adding to antimicrobial activity (e.g., EDTA, GLDA). Some pH balancers may be multi-functional by adding to antimicrobial activity (e.g., some acids, such as citric acid, glutamic acid, N, N-diacetic acid, lactic acid, phytic acid).

A chelating agent (chelant) herein, when included, is or may be derived from at least one of a group provided herein, or may be a functional and/or chemical equivalent therein, functionally active and/or chemically active derivatives thereof, and/or functionally active and/or chemically active analogs thereof, understood by one of skill in the art as acting as a chelating agent for a skin and/or hair composition or formulations herein. Representative, and non-limiting examples of chelating agent include, but are not limited to, agents (molecules or compounds) that bind, “chelate,” and/or sequester metal ions (e.g., calcium, magnesium, manganese, iron, nickel, chromium, copper, as examples), and then form stable complexes with the metal ions (which may alter the electronic charge of the metal ion). Illustrative and further representative examples of a chelating agent include, but are not limited to: diiospropyl oxalate, disodium ethylene diamine tetraacetic acid (EDTA), disodium EDTA-copper, EDTA, HEDTA, oxalic acid, potassium citrate, sodium citrate, tetrahydroxypropyl ethylenediamine, sodium oxalate, TEA-EDTA, tetrasodium EDTA, trisodium EDTA, trisodium HEDTA, nitrilotriacetic acid (NTA), iminodisuccinate (IDS), citric acid, sodium gluconate, ethylenediamine-N,-N-disuccinic acid (EDDA), ethylenediaminedisuccinic acid (EDDS), L-glutamic acid N,N-diacetic acid tetrasodium salt (GLDA), methyl glycinediacetic acid (MGDA), ethylenediamine N,N′-diglutaric acid (EDDG), ethylenediamine-N,N-diamalonic acid (EDDM), 2-hydroxyethyliminodiacetic acid (HEIDA), 3-hydroxy-2,2-iminodisuccinic acid (HIDS), pyridine-2,6-dicarboxylic acid (PDA), silicates, polymers of acrylic and maleic acid, and tetrasodium glutamate diacetic acid (in their acetate or acid form), and analogs or derivatives thereof having activity as a chelating agent. Representative products for natural, and/or biodegradable chelating agents include HIDS™ (a tradename applied for by Nippon Shokubai Co, LTD. Tokyo, Japan, for HIDS or tetrasodium 3-hydroxy-2,2′-iminodisuccinate), Dissolvine® (a trademark registered to Akzo Cheme Nederlands B.V. Amersfoort, Netherlands, for GLDA or glutamic acid, N,N-diacetic acid, tetrasodium salt), and Trilon® M (a trademark registered to Badische Anilin-& Soda-Fabrik Aktiengesellschaft Corporation, Rhine, Fed. Rep. Germany, for MGDA or methylglycinediacetic acid trisodium salt). In one or more embodiments, a chelating agent is or includes a natural, and/or biodegradable chelating agent. In one or more embodiments, a chelating agent is or includes one or more of an EDTA (or forms thereof), NTA, IDS, GLDA, MGDA, EDDA, EDDG, EDDM, EDDS, HIDS, HEIDA, or PDA or combinations thereof. The amount of a chelating agent in a composition or formulation herein will be from about 0.05 wt. % to about 4 wt. %. In some embodiments, the amount of the chelating agent in a ready to use composition and/or formulation will be from about 0.1 wt. % to about 2 wt. %. In some embodiments, an amount of the chelating agent will be from about 0.05 wt. % to about 1 wt. %. In some embodiments, an amount of the chelating agent will be from about 0.1 wt. % to about 1 wt. %. In some embodiments, an amount of the chelating agent will be from about 0.5 wt. % to about 2 wt. %. In some embodiments, an amount of chelating agent in a ready-to-use composition and/or formulation will be up to about 2 wt. %. In some embodiments, an amount of chelating agent in a ready-to-use composition and/or formulation will be up to about 1 wt. %. In some embodiments, the amount of chelating agent will be up to about 0.5 wt. %. The amount is based on a total weight of the composition and/or formulation, and/or may be in any range there between.

A humectant herein, when included, is or may be derived from at least one of a group provided herein, or may be a functional and/or chemical equivalent therein, functionally active and/or chemically active derivatives thereof, and/or functionally active and/or chemically active analogs thereof, understood by one of skill in the art as acting as a humectant for a skin and/or hair composition and/or formulation herein. In one or more forms, a humectant will reduce “stickiness” associated with the APG when utilized in the amounts or concentrations described herein. A humectant as described herein will impart better workability of the composition and/or formulation described herein, so as to improve distribution of the composition and/or formulation when prepared as a product for applying to skin and/or hair and/or for distributing on or through skin and/or hair. Representative, and non-limiting examples of a humectant for compositions and/or formulations described herein include, but are not limited to, substances (e.g., molecules or compounds) that may be mixed and/or emulsified with water, thus attract and/or bind water. In one or more embodiments, the humectant may behave like a humectant, and has a fatty acid component and may be a lipid, or have a lipid-like arrangement. Illustrative and further representative examples of a humectant for compositions and/or formulations herein include, but are not limited to: tristearin, glycerin, glyceryl stearate, triglyceryl stearate, sorbitol, butylene glycol, 1,3-propanediol (a plant-based alternative to propylene glycol), diethylene glycol, caprylyl glycol, glycine betaine, grape seed oil, lanolin, acetylated lanolin, honey, salt form of pyrrolidine carboxylic acid (pyroglutamic acid or sodium PCA). In certain embodiments, the humectant is a natural ingredient, originating in whole or in part from, made in whole or in part from and/or extracted in whole or in part from a vegetable oil or fruit oil, exemplified by but not limited to coconut, coconut oil, soybean, soybean oil, palm, palm oil, corn, corn oil, corn syrup, sugar cane, beet sugar, vegetable oil, hydrolyzed wheat (and wheat proteins), hydrolyzed oat (and oat proteins), baobab, and aloe vera. In some embodiments, the humectant is or includes one or more of glycerin or glycerol or sorbitol or caprylyl glycol or diethylene glycol or 1,3-propanediol. In one or more embodiments, the humectant is or includes one or more of honey or aloe vera gel or liquid. The amount of a humectant in a composition and/or formulation herein will be from about 0.1 wt. % to about 7 wt. %. In some embodiments, the amount of the humectant will be from about 0.1 wt. % to about 5 wt. %. In some embodiments, the amount of the humectant will be from about 0.5 wt. % to about 5 wt. %. In some embodiments, the amount of the humectant will be up to about 5 wt. %. In some embodiments, the amount of the humectant will be up to about or at about 4 wt. %. In some embodiments, the amount of the humectant will be up to about or at about 3 wt. %. In some embodiments, the amount of the humectant will be up to about or at about 2 wt. %. The amount is based on a total weight of the composition and/or formulation, and/or may be in any range there between.

In some embodiments, a humectant may be replaced by a ceramide as is understood and used for hair and/or skin products as a lipid or sphingosine or sphingolipid metabolite (generally having a sphingoid base), and the ceramide will be in ranges and amounts described above for a humectant. In other embodiments, a humectant may be replaced by an emollient being a natural oil or natural butter. Natural oils are liquid at room or ambient temperature having a melting point below 20 degrees Centigrade; natural butters have a melting point from 20 to about 40.5 degrees Centigrade. The emollient will be in ranges and amounts described above for a humectant. A composition and/or formulation having a ceramide rather than a humectant will add lipids and fatty acids to the hair and/or skin and moisturize as well as protect the hair and/or skin from drying. In a composition and/or formulation having an emollient rather than a humectant, the emollient will moisturize and add oil to the hair and/or skin to smooth and/or repair the hair and/or skin.

A SAC agent herein, when included, helps aid in stability of an emulsification formed when components are provided in a composition and/or formulation herein, and prevents separation and/or disruption and/or particulation (particle formation and/or flocculation and/or sedimentation) of a final composition and/or formulation. A SAC agent may be a first SAC agent, and include functional and/or chemical equivalent therein, functionally active and/or chemically active derivatives thereof, and/or functionally active and/or chemically active analogs thereof, as is understood by one of skill in the art as acting to help solubilize and/or stabilize the one or more APG and/or strengthen the emulsion for a skin and/or hair composition and/or formulation herein. Representative, and non-limiting examples include, but are not limited to, a secondary surfactant or substance (e.g., molecule or compound) that acts similar to or is a non-ionic surfactant. Illustrative and further representative examples include, but are not limited to one or more of: polysorbate (e.g., polysorbate 20, polysorbate 80), fatty acid alkanolamides (e.g., lauric monoethanolamide, diethanolamide, cocamide), decyl glucoside, coco glucoside, caprylyl/capryl glucoside, polyethylene glycol (PEG)-50 shea butter, olive oil PEG-7 esters, coco betaine, and sulfated castor oil. The amount of a SAC agent in a ready-to-use composition and/or formulation described herein will be from about 0.5 wt. % to about 10 wt. %. In some embodiments, the amount will be from about 1 wt. % to about 8 wt. %. In some embodiments, the amount will be from about 2 wt. % to about 8 wt. %. In some embodiments, the amount will be from about 3 wt. % to about 5 wt. %. In some embodiments, the amount will be from about 1 wt. % to about 5 wt. %. In some embodiments, the amount will be less than about 8 wt. %. In some embodiments, the amount will be less than about 7 wt. %. In some embodiments, the amount will be less than about 6 wt. %. In some embodiments, the amount will be less than about 5 wt. %. In some embodiments, the amount will be about 4 wt. %. The amount is based on a total weight of the composition and/or formulation, and/or may be in any range there between.

A second SAC agent may optionally be included in one or more embodiments, to further aid in emulsion stability, to help clarify the emulsion of a final composition and/or formulation. In one or more embodiments, a second SAC agent is mild when included, working cooperatively with a first SAC agent. It is understood that a first SAC agent may be included in a composition and/or formulation herein, with or without addition of a second SAC agent. Representative, and non-limiting examples of a second SAC agent for compositions and/or formulations herein include, but are not limited to one or more of: fatty alkanolamides, and alkyl taurate amides and taurate salts having a same taurate core (sulfate-free mild emulsifying agents), such as sodium methyl cocoyl taurate, potassium taurate, sodium methyl taurate, sodium taurate, sodium lauroyl taurate, calcium lauroyl taurate, sodium methyl lauroyl taurate, magnesium methyl cocoyl taurate, potassium methyl cocoyl taurate, sodium methyl oleoyl taurate, sodium methyl myristoyl taurate, sodium methyl palmitoyl taurate, sodium methyl stearoyl taurate, sodium taurine cococyl methyl taurate, sodium n-isosearoyl methyltaurate, sodium methyltaurine cococyl methyltaurate, and sodium taurine cocoyl methyltaurate, as representative examples. In addition, or as a further alternative, suitable in one or more embodiments, a mild second SAC agent may include, as non-limiting examples, one or more of: sodium cocoyl glutamate, sodium laureth-13 carboxylate, sodium cocoyl isethionate, alkyl methyl glucamide, lauroyl methyl glucamide, cocoyl methyl glucamide, myristoyl methyl glucamide, capryloyl/caporyl methyl glucamide, and sodium cocoyl glycinate. Combined, a first SAC agent and second SAC agent will be in an amount described above for a SAC agent in a ready-to-use composition and/or formulation (e.g., from about 0.5 wt. % to about 10 wt. % or any range there between).

A conditioning agent herein, when included, is or may be derived from at least one of a group provided herein, or may be a functional and/or chemical equivalent therein, functionally active and/or chemically active derivatives thereof, and/or functionally active and/or chemically active analogs thereof, understood by one of skill in the art as acting as a conditioning agent for a skin and/or hair composition and/or formulation herein. Representative, and non-limiting examples of a conditioning agent for compositions and/or formulations herein include, but are not limited to, substances (e.g., molecules or compounds) that form a layer and help smooth a surface and/or may thicken a composition and/or formulation herein. Illustrative and further representative examples of a conditioning agent include, but are not limited to: one or more phospholipids derived from an oil from a natural source or a vegetable. Generally, the oil may be from a vegetable, seed, plant, root, or fruit. Suitable oils include, but are not limited to soybean oil, wheat germ oil, rapeseed oil, coconut oil, and sunflower seed oil. The vegetable may include, but is not limited to soybeans, rape seed, wheat germ, sunflower, and flax seed. Further examples of a conditioning agent as used herein include coconut oil phospholipids, cocamidopropyls, dicaprylyl carbonate, and cocamidopropyl PG-dimonium chloride phosphate, and lecithin. A representative product as a natural conditioning agent is Cola® Lipid C (a tradename registered with Colonial Chemical Co, Tennessee, USA, for a phosphoric acid, triester with N-(2,3-dihydroxypropyl)-N,N-dimethyl-3-[(1-oxococo-alkyl)amino-1-propanaminium chloride). The amount of a conditioning agent in a composition and/or formulation described herein will be from about 0.05 wt. % to about 10 wt. %. In some embodiments, the amount will be from about 0.5 wt. % to about 8 wt. %. Or, the amount herein will be from about 1 wt. % to about 8 wt. %. In some embodiments, the amount will be from about 1 wt. % to about 5 wt. %. In some embodiments, the amount will be up to about 10 wt. %. In some embodiments, the amount will be up to about 9 wt. %. In some embodiments, the amount will be up to about 8 wt. %. In some embodiments, the amount will be up to about 8 wt. %. In some embodiments, the amount will be up to about 6 wt. %. In some embodiments, the amount will be up to about 5 wt. %. In some embodiments, the amount will be up to about 3 wt. %. In some embodiments, the amount will be up to about 2 wt. %. In some embodiments, the amount will be up to about 1 wt. %. The amount is based on the total weight of the composition and/or formulation, and/or may be in any range there between.

The tables below provide exemplary and suitable ranges for each of the components that may be utilized in one or more multi-functional compositions and/or formulations described herein, ones that will remain stable with storage, including those with APG at high concentrations (higher than expected to remain stable) and in a lower pH (neutral or acidic). The amounts or ranges for each component are rounded to a nearest whole number and are representative, provided as wt. %, based on total composition weight. The representative ranges are those that may be utilized (at or at about an amount in each range) for preparation of a useful composition and/or formulation that when finally prepared may be utilized for parasite control, removal, cleansing, prevention and/or treatment, and/or conditioning of hair and/or skin of a subject in need thereof, said subject having or containing or suspected of having or containing a parasite or an infestation of a parasite, the parasite being one or more of a biting and/or feeding parasite, selected from any one or more of a family of lice, biting flies, fleas, bedbugs, mites, scabies, ticks, and the like, such as described herein. For more concentrated forms, water is removed. Compositions and/or formulations described herein work best when ratios of each of the components are maintained, said ratios being in accordance with ranges of each component as described above or as illustrated in at least TABLE 1. The first and/or the second SAC agent, when included, are provided as independent (separate) agents. The optional components and/or further additives, which may or may not be included, and when added, are at the expense of water.

TABLE 1

| COMPONENTS OF COMPOSITION/FORMULATION | wt. % |
|---|---|
| alkyl polyglucoside(s) | >12-50 |
| co-solvent or carrier for water | 1-20 |
| pH balancer | 0.05-5 |
| humectant(s) | 0.05-10 |
| chelating agent | 0.05-5 |
| antimicrobial/preservative(s) | 0.05-3 |
| first solubilizing and clarifying agent (optional) | 0-10 |
| second solubilizing and clarifying agent (optional) | 0-5 |
| conditioning agent (optional) | 0-5 |
| further additive as thickener (optional) | 0-5 |
| further additive as fragrance (essential oil(s) (optional) | 0-5 |
| further additive as enzyme(s) (optional) | 0-13 |
| water (balance) | 0.1-90 |

TABLE 2 provides illustrative examples of proportionally adjusting amounts of components in compositions and/or formulations described herein, ones that will remain stable with storage, particularly with APG at high concentrations (higher concentration than expected to remain stable) and in a lower pH (neutral or acidic). In TABLE 2, while amounts of various components have been adjusted, ratios are proportional. Taking A-4 of TABLE 2 as an example, each of A-3, A-2, and A-1 are proportionally less than A-4 by approximately 10% between each of A-3, A-2, and A-1, respectively. Similarly, each of A-5, A-6, and A-7 of TABLE 2 are proportionally greater than A-4 by approximately 10% between each of A-5, A-6, and A-7, respectively. And when non-water components are raised (proportionally) for a more concentrated form, water content is decreased. It is noted that a co-solvent for water is not adjusted proportionally due to its requirement in a fixed amount or overall ratio.

TABLE 2

| | A-1 | A-2 | A-3 | A-4 | A-5 | A-6 | A-7 |
|---|---|---|---|---|---|---|---|
| | wt. % (based on total composition) | | | | | | |
| alkyl polyglucoside(s) | 18-25 | 20-28 | 22-32 | 25-35 | 27-39 | 30-42 | 33-47 |
| co-solvent/carrier | 5-15 | 5-15 | 5-15 | 5-15 | 5-15 | 5-15 | 5-15 |
| humectant(s) | 0.07-2.2 | 0.08-2.4 | 0.09-2.7 | 0.1-3 | 0.11-3.3 | 0.12-3.6 | 0.13-4 |
| chelating agent | 0.07-0.73 | 0.08-0.81 | 0.09-0.9 | 0.1-1 | 0.11-1.1 | 0.12-1.2 | 0.13-1.3 |
| pH balancer | 0.07-0.73 | 0.08-0.81 | 0.09-0.9 | 0.1-1 | 0.11-1.1 | 0.12-1.2 | 0.13-1.3 |
| antimicrobial/ | 0.02-1.1 | 0.03-1.3 | 0.04-1.4 | 0.05-1.5 | 0.06-1.7 | 0.07-1.9 | 0.08-2.0 |
| preservative(s) | | | | | | | |
| first and/or second | 0-3.8 | 0-4 | 0-4.5 | 0-5 | 0-5.5 | 0-6 | 0-6.7 |
| SAC (optional) | | | | | | | |
| conditioning agent | 0-1.4 | 0-1.6 | 0-1.8 | 0-2 | 0-6 | 0-7 | 0-8 |
| (optional) | | | | | | | |
| water (for ready-to- | 59-80 | 54-73 | 49-66 | 45-60 | 40-54 | 36-49 | 32-44 |
| use or concentrate) | | | | | | | |

TABLE 3 illustrates further representative ready-to-use compositions and/or formulations, which, when finally prepared, for utilization with a subject in need having or containing or suspected of having or containing a parasite or an infestation of a parasite (e.g., one or more of lice, biting flies, fleas, bedbugs, mites, scabies, ticks, and the like). The representative compositions will remain stable with storage, particularly with APG at high concentrations (higher concentration than expected to remain stable) and in a lower pH (neutral or acidic).

TABLE 3

| | R-1 | R-2 | R-3 | R-4 | R-5 |
|---|---|---|---|---|---|
| READY-TO-USE (R) | wt. % (based on total composition) | | | | |
| alkyl polyglucoside(s) | 25-35 | 20-30 | 16-22 | 12.5-17 | 31-44 |
| co-solvent for water | 5-16 | 6-18 | 7-18 | 8-18 | 3-13 |
| humectant(s) | 0.1-3 | 0.08-2.5 | 0.06-1.9 | 0.04-1.6 | 0.125-3.8 |
| chelating agent | 0.1-1 | 0.08-0.8 | 0.06-0.6 | 0.04-0.4 | 0.125-1.25 |
| pH balancer | 0.1-1 | 0.08-0.8 | 0.06-0.6 | 0.04-0.4 | 0.125-1.25 |
| antimicrobial/preservative(s) | 0.1-1.5 | 0.08-1.2 | 0.06-1 | 0.04-0.8 | 0.125-1.9 |
| first and/or second SAC agent | 0-6 | 0-5 | 0-4 | 0-4 | 0-6.3 |
| (optional) | | | | | |
| conditioning agent (optional) | 0-2 | 0-1.6 | 0-1.3 | 0-1 | 0-2.5 |
| water (balance) | 45-60 | 54-72 | 64-87 | 76-95+ | 33-45 |

TABLE 4 provides further illustrative examples of proportionally adjusting amounts of components in compositions and/or formulations described herein, which are generally in the form of a concentrate, in which a concentration of APG is greater than about 50% based on the total composition. These will remain stable with storage, particularly with APG at high concentrations (higher concentration than expected to remain stable) and in a lower pH (neutral or acidic). Said concentrates may be diluted with sufficient water to form ready-to-use compositions and/or formulations as described herein.

TABLE 4

| | C-1 | C-2 | C-2 | C-4 |
|---|---|---|---|---|
| CONCENTRATE (C) | wt. % (based on total composition) | | | |
| alkyl polyglucoside(s) | 51-55 | 56-60 | 61-65 | 66-70 |
| co-solvent for water | 2.25-7.5 | 2-7 | 1.8-6.5 | 1.6-5.5 |
| humectant(s) | 1.6-4.7 | 0.17-5.2 | 0.18-5.8 | 0.2-6.5 |
| chelating agent | 0.16-1.6 | 0.17-1.7 | 0.18-1.8 | 0.19-1.9 |
| pH balancer | 0.16-1.6 | 0.17-1.7 | 0.18-1.8 | 0.19-1.9 |
| antimicrobial/ | 0.16-2.3 | 0.17-2.5 | 0.18-2.8 | 0.19-3 |
| preservative(s) | | | | |
| first and/or second | 0-7.5 | 0-8.5 | 0-9 | 0-10 |
| SAC agent (optional) | | | | |
| conditioning agent | 0-3 | 0-3.3 | 0-3.7 | 0-4.1 |
| (optional) | | | | |
| water (balance) | 20-27 | 18-24 | 16-22 | 14-20 |

Laboratory tests evaluating R-1 and R-2 as compositions/formulations found both R-1 and R-2 to be effective, individually, against head lice when the APG was a lauryl glucoside, or contained a combination of lauryl glucoside and other alkyl glucoside(s) (in which a lauryl glucoside was the only APG, or a lauryl glycoside was a predominant APG and comprised greater than 50% of the total alkyl glucosides present in the APG component). The head lice (as well as nits and eggs) were eliminated when such a composition/formulation was an R-1 composition/formulation or an R-2, after either an R-1 or an R-2 was applied to head lice (in only one application). It was found that an R-1 and an R-2 had a coating effect on the insect, immobilizing the insect and suffocating the insect. It was also found that both R-1 and R-2 dehydrated the outer waxy coating of the head lice. Similar laboratory tests evaluating a same R-1 and a same R-2, each utilized individually, found each of R-1 and R-2 were less effective against fleas when the APG was a lauryl glucoside, or contained a combination of lauryl glucoside and other alkyl glucosides (in which lauryl glucoside was predominant and comprised greater than 50% of the total alkyl glucosides present as the APG component). While greater than 60% of fleas were eliminated, not all fleas were eliminated. It is recommended that more than one application of a composition/formulation containing lauryl glucoside (alone or as a predominant APG component) should be utilized against fleas, since more than one application will perform better and should completely eliminate fleas.

When evaluating an R-1 and an R-2 in laboratory tests against fleas and when the APG was a decyl glucoside (alone or in an amount greater than 50% of the total alkyl glucosides present as the APG component), both an R-1 and an R-2, when applied to fleas, were effective against fleas, eliminating the fleas (and eggs and residue) in one application. A same R-1 or a same R-2 containing decyl glucoside as the predominant APG (alone or in an amount greater than 50% of the total alkyl glucosides present as the APG component) was not as effective, when utilized individually against head lice (although a majority of head lice were eliminated in a manner akin to that described above, not all were eliminated, which is contrary to what was found when the APG component was lauryl glucoside or predominantly lauryl glucoside). It is recommended that more than one application of a composition/formulation containing decyl glucoside (alone or as a predominant APG component) should be utilized against lice, since more than one application will perform better and possibly completely eliminate head lice. Alternatively, to combat both fleas and lice, a combination of a lauryl glucoside and decyl glucoside (in any forms described above) in an R-1 or R-2 formulation may be provided.

When evaluating an R-1 composition/formulation or an R-2 composition/formulation individually in which the APG component was a coco glucoside (coconut based, and predominantly C8-C14 alkyl glucosides, in which greater than 50% of the total alkyl glucosides present in the APG coco glucoside component are generally C12 and C14), both R-1 and R-2 were found to be effective, individually, against head lice. The head lice were eliminated in a similar manner as described above, when such an R-1 composition/formulation or an R-2 compositions/formulations was applied to head lice (in one application). Similar laboratory tests evaluating the R-1 compositions/formulations and the R-2 compositions/formulations, individually, found R-1 and R-2 were each less effective against fleas when the APG was a coco glucoside. While more than 60% of fleas were eliminated, not all fleas were eliminated. It is likely that more than one application of a compositions/formulations containing coco glucoside as the APG component would perform better and possibly completely eliminate fleas. Alternatively, to combat both fleas and lice, a combination of a lauryl glucoside and coco glucoside (in any forms described above) in an R-1 or R-2 formulation may be provided. It is recommended that more than one application of a composition/formulation containing coco glucoside (alone or as a predominant APG component) should be utilized against fleas, since more than one application will perform better and possibly completely eliminate fleas. Alternatively, to combat both fleas and lice, a combination of a decyl glucoside and coco glucoside (in any forms described above) in an R-1 or R-2 formulation may be provided.

R-1 ready-to-use compositions/formulations and R-2 ready-to-use compositions/formulations evaluated in the above exemplary laboratory tests, will contain, in addition to the APG component, components of water, a co-solvent for water, a chelating agent, a humectant, a pH balancer, and antimicrobial/preservatives. Some of the R-1 ready-to-use compositions/formulations and R-2 ready-to-use composition/formulation evaluated in the above exemplary laboratory tests contained, in addition to the APG component, water, a co-solvent for water, a chelating agent, a humectant, a pH balancer, antimicrobial/preservatives, and a SAC agent. R-1 and R-2 compositions achieved stability of its APG when provided at a high concentration (higher concentration than expected to remain stable) and in a lower pH, and maintained handleability and split suppression, especially after storage.

Exemplary R-1 or R-2 compositions/formulations included a lauryl glucoside or a decyl glucoside or a coco glucoside [as the APG, supplied by a well-known and reputable supplier, such as BASF®, which presently supplies at least Plantaren® 1200 N UP (lauryl glucoside), Plantaren® 1200 N (lauryl glucoside), Plantaren® 1300 N (lauryl glucoside), Plantaren® 818 UP (coco glucoside), Plantaren® 2000 N (decyl glucoside), Plantaren® 2000 N UP (decyl glucoside), Plantaren® 2000 N UP/MB (decyl glucoside), Plantacare® 1200 UP (lauryl glucoside), Plantacare® 818 UP (coco glucoside), Plantacare® 2000 UP (decyl glucoside) (BASF is registered with BASF SE Societas Europae (SE) Fed. Rep. Germany, Carl-Bosch-Strasse 38 Ludwigshafen Am. Rhein, Fed. Rep. Germany 67056; Plantaren® and Plantacare® and Plantapon® are registered with Cognis IP Management GMBH Corporation Fed. Rep. Germany, Henkelstrasse 67 Duesseldorf, Fed. Rep. Germany D-40191]. However, the APG or APG combinations may also be one or more of: lauryl glucoside (C12 alkyl glucoside, or any combination of alkyl glucosides generally from C10-C14, with a predominance of C12 and C14 and lauryl glucoside being in an amount that is greater than 50%); coco glucoside (coconut based, and predominantly C8-C14 alkyl glucosides, and in which greater than 50% of the total alkyl glucosides present in the APG coco glucoside component are generally C12 and C14, or provided as a combination of at or about 40% C8-C10 alkyl glucosides and at or about 60% C12-C16 alkyl glucosides); coco glucoside (coconut based, and predominantly C8-C14 alkyl glucosides, with C8-C10 alkyl glucosides<C12-C14 alkyl glucosides); decyl glucoside (coconut based, and predominantly C8-C14 alkyl glucosides, and in which greater than 50% of the total alkyl glucosides present in the APG decyl glucoside component are generally C8 and C10, or provided as a combination of at or about 60% C8-C10 alkyl glucosides and at or about 40% C12-C16 alkyl glucosides); decyl glucoside (coconut based, and predominantly C8-C14 alkyl glucosides, with C8-C10 alkyl glucosides>C12-C14 alkyl glucosides); dodecyl D-glucoside (provided as a combination of at or about 40% C8-C10 alkyl glucosides and at or about 60% C12-C14 alkyl glucosides), as preferred but non-limiting examples.

For the exemplary R-1 and in R-2 compositions/formulations, the co-solvent for water was a medical grade polyethylene glycol having a low average number of ethylene glycol units (e.g., PEG-8, which may be substituted with another suitable low average number of ethylene glycol units from about 4 ethylene glycol units to about 200 ethylene glycol units, as preferred examples, and generally not greater than about 400 ethylene glycol units). In some embodiments, a co-solvent for water is a polyethylene glycol having a low average number of ethylene glycol units (e.g., less than 400 ethylene glycol units, or 200 ethylene glycol units or less).

For the exemplary R-1 and in R-2 compositions/formulations, the humectant was glycerin of a vegetable origin. In some of the evaluated R-1 and in R-2 compositions/formulations, the humectant was caprylyl glycol. In some of the evaluated R-1 and in R-2 compositions/formulations, the humectant was 1,3-propanediol (provided as NatureSilk, CAS 504-63-2, a plant-based alternative to propylene glycol). In some of the evaluated R-1 and in R-2 compositions/formulations, the humectant was diethylene glycol. In some of the evaluated R-1 and in R-2 compositions/formulations, the humectant was glycerin of a vegetable origin and caprylyl glycol. In one or more preferred embodiments, the humectant is made from coconut oil, soy oil, or palm oil.

For the exemplary R-1 and in R-2 compositions/formulations, the pH balancer was citric acid. Citric acid may be substituted with humic acid, lactic acid, or hydrochloric acid, as some preferred but non-limiting examples.

For R-1 and in R-2, the chelating agent was tetra sodium glutamate diacetate. The tetra sodium glutamate diacetate may be substituted with disodium EDTA, trisodium EDTA, tetrahydroxypropyl ethylenediamine, or tetra sodium EDTA, including Dissolvine® chelates (supplied by Nouryon®), such as but not limited to Dissolvine® GL Plus (glutamic acid, N,N,-diacetic acid, tetrasodium salt), Dissolvine® GL-47-S (glutamic acid, N,N,-diacetic acid, tetrasodium salt), Dissolvine® GL-PD-S (glutamic acid, N,N,-diacetic acid, tetrasodium salt), Dissolvine® H-40 (hydroxyethylenediaminetriacetic acid, trisodium salt), Dissolvine® H-88-X (hydroxyethylenediaminetriacetic acid, trisodium salt), Dissolvine® NA (EDTA, tetrasodium salt), Dissolvine® NA-X (EDTA, tetrasodium salt, tetrahydrate), Dissolvine® 100-S (EDTA, tetrasodium salt), Dissolvine® 105-E (EDTA, tetrasodium salt), Dissolvine® E-39 (EDTA, tetrasodium salt), Dissolvine® 220-S (EDTA, tetrasodium salt, tetrahydrate), Dissolvine® NA2 (EDTA, disodium salt), Dissolvine® NA2-S (EDTA, disodium salt, dihydrate), Dissolvine® NA2-P (EDTA, disodium salt, dihydrate), Dissolvine® NA3-36 (EDTA, trisodium salt), (Nouryon is registered with Nouryon Chemicals International B.V., Velperweg 76 Arnhem Netherlands 6824 BM; and Dissolvine is registered with AKZO Nobel Chemicals B.V. LLC, Velperweg 76 Arnhem Netherlands 6824 BM).

For the exemplary R-1 and in R-2 compositions/formulations, the antimicrobial/preservative was phenoxyethanol from a vegetable source (alone or in combination with other preservatives to reduce eye irritation) in combination with isopentyldiol. The pH balancer may also be one or more of phenoxyethanol potassium sorbate, a paraben, an isothiazolinone, benzoic acid, triclosan, glycol ether, benzalkonium chloride, glyceryl caprilate, ethyl lauroyl arginate, isopentyldiol, and phenoxyethanol.

For the exemplary R-1 and in R-2 compositions/formulations, when the SAC agent was included, the SAC agent was polysorbate 20, which may be replaced by any of coco glucoside, decyl glucoside, a taurate salt.

For utilization of a composition and/or formulation described herein (or product therefrom), it is applied to dry hair and/or skin. A ready-to-use form should require no further water or dilution. For better efficacy and results as an insecticide (anti-parasitic), a ready-to-use form should not be diluted.

TABLE 5 shows other representative ready-to-use multi-function compositions and/or formulations that will remain stable with storage, maintaining split suppression and handleability with APG at high concentrations (higher concentration than expected to remain stable) and in a lower pH (neutral or acidic). For TABLE 5, the APG is a glucoside from coconut or corn (e.g., Plantaren® 1200), with purified and/or distilled water, polyethylene glycol (e.g., PEG 8) as the co-solvent or carrier for water, glycerin from vegetable and/or caprylyl glycol as the humectant, citric acid as the pH balancer, tetrasodium glutamate diacetate from vegetable (e.g., Dissolvine® GL 47-S) as the chelating agent, phenoxyethanol and isopentyldiol as antimicrobial/preservatives (e.g., Argan-o-cide PE and isopentyldiol), with or without a coconut oil derived phospholipid (e.g., Cola® Lipid C) as a conditioning agent. In some embodiments, both glycerin from vegetable and caprylyl glycol are included.

TABLE 5

| | R-6 | R-7 | R-8 | R-9 |
|---|---|---|---|---|
| READY-TO-USE (R) | wt. % (based on total composition) | | | |
| alkyl polyglucoside(s) | 21-30 | 25-35 | 15-35 | 20-40 |
| co-solvent for water | 4-15 | 5-18 | 3-18 | 4-15 |
| humectant(s) | 0.4-5 | 0.5-5 | 0.1-5 | 0.4-5 |
| chelating agent | 0.08-1.8 | 0.1-2 | 0.06-2 | 0.08-2 |
| pH balancer | 0.08-1.8 | 0.1-2 | 0.06-2 | 0.08-2 |
| antimicrobial/preservative(s) | 0.08-1.8 | 0.1-2 | 0.06-2 | 0.08-2 |
| conditioning agent | 0-2.5 | 0-3 | 0.3-3 | 0.4-5 |
| water (balance) | 28-60 | 33-70 | 33-80 | 25-75 |

TABLE 6 provides other representative multi-functional compositions and/or formulations and suitable ranges for components, in which the composition will remain stable with storage in a lower pH (neutral or acidic). For TABLE 6, propanediol from corn was the co-solvent or carrier for water, caprylyl glycol from coconut was the humectant, phenoxyethanol (e.g., Argan-o-cide) was the antimicrobial/preservative, and either one or both of a coconut oil derived phospholipid (e.g., Cola® Lipid C) and/or dicaprylyl carbonate from vegetable was/were the conditioning agent (or can also use phosphoric acid triester with N-(2,3-dihydroxypropyl)-N,N-dimethyl-3-[(1-oxococo-alkyl)amino-1-propanaminium chloride or cocamidopropyl PG-cimonium chloride phosphate or natural triglyceride). An SAC agent, when provided, is polysorbate 20 and/or decyl glucoside and/or coco glucoside and/or sodium methyl cocoyl taurate. Fragrance when provided is often essential oil(s), for example a proprietary blend containing lavender, rosemary leaf, lemon, eucalyptus, and lemongrass, or one or some essential oils, all of which are selected for repellence properties. The ready-to-use stable compositions/formulations from TABLE 7 may be provided as a spray or spritz (S-1, S-2), or in more concentrated forms (S-3, S-4). Testings on working compositions of TABLES 6 and 7 showed they repelled parasites, being best utilized for styling and/or repellence, either as a comb-out/brush-out product or as leave-in product (data not shown). These products have an added benefit as a detangler, allowing for easy combing/brushing of all hair types for ease in removal of lice, fleas, mites, eggs, nits, as examples. In some embodiments, compositions of TABLES 6 or 7 may be utilized before and/or after any composition of TABLES 1-5 or 8-11. Any of such combinations may be used daily or part of a normal hair and/or skin cleansing and conditioning routine.

TABLE 6

| | wt. % (based on total |
|---|---|
| | composition) |
| co-solvent or carrier for water | 0.05-5 |
| humectant | 0.1-3 |
| antimicrobial/preservative(s) | 0.05-3 |
| conditioning agent(s) | 0.5-6 |
| solubilizing and clarifying agent(s) (optional) | 0-5 |
| fragrance (optional) | 0-1 |
| water (balance) | 80-99.5 |

TABLE 7

| | S-1 | S-2 | S-3 | S-4 |
|---|---|---|---|---|
| | wt. % (based on total composition) | | | |
| co-solvent or carrier for water | 0.3-3 | 0.5-5 | 1-8 | 2-16 |
| humectant | 0.05-2 | 0.1-3 | 0.5-5 | 1-10 |
| antimicrobial/preservative(s) | 0.05-2 | 0.1-3 | 0.5-5 | 1-10 |
| conditioning agent(s) | 0.3-2 | 0.5-3 | 1-5 | 2-10 |
| fragrance | 0.03-0.6 | 0.05-1 | 0.05-1.6 | 1-3.5 |
| water (balance) | 90-99 | 85-99 | 75-97 | 50-93 |

TABLES 8 and 9 provide yet further representative multi-functional compositions and/or formulations with APG, showing suitable ranges for each of the components, utilized as a ready-to-use shampoo or shampoo mousse or spray or spritz for hair and/or skin that will remain stable with storage, maintaining split suppression and handleability with APG at high concentrations (higher concentration than expected to remain stable) and in a lower pH (neutral or acidic). The component ranges are rounded to a nearest whole number and are representative. The compositions and/or formulations of TABLES 8 and 9 have no added color, no added fragrance, no animal ingredients, and no pesticides, and all ingredients are considered cruelty free. More concentrated forms for compositions and/or formulations of TABLE 8 and TABLE 9 may be prepared, in which water is at least 25% of the final composition, and all other components are increased proportionally to what is disclosed in TABLE 8 or TABLE 9. The shampoo or shampoo mousse forms of TABLES 8 and 9 are effective for cleansing and/or conditioning hair and/or skin while also being effective against parasites, by physical or chemical action, for control, removal, prevention, and/or for treatment against one or more biting and/or feeding parasites and/or by-products thereof (e.g., having a killing and knock down effect when utilized on the one or more parasites), the parasites being one or more of lice, fleas, bedbugs, mites, scabies, ticks, and the like.

Compositions/formulations of TABLES 8 and 9 include: a lauryl glucoside (which may be provided, e.g., as Plantaren® 1200 N) or a decyl glucoside (e.g., Plantarenº 2000) as the APG; GLDA (glutamic acid, N,N-diacetic acid, tetrasodium salt, which may be provided as Dissolvine® GL-47-S) as the chelating agent; polyethylene glycol (which may be, e.g., Pluracare® E 400 or Pluracare® E 400 NF or Pluracare® E 400 NF PEG-FLEX; Pluracare® being registered with BASF Corporation, New Jersey, USA) as co-solvent or carrier for water; glycerin from vegetable as the humectant; phenoxyethanol with capryl glycol and isopentyldiol (in which phenoxyethanol may be provided, e.g., as Argan-o-cide PE) as antimicrobials/preservatives; polysorbate 20 (which may be provided, e.g., as Eumulgin® SML 20, registered with BASF Corporation, New Jersey, USA) as the first SAC agent; sodium methyl cocoyl taurate as the second SAC agent; and citric acid as the pH balancer (to bring the pH to less than 7.0, or at or about pH 6.8). Water is provided as filtered water. In some embodiments, palm oil is a source for the lauryl glucoside. In some embodiments, coconut and/or coconut oil are a source for ingredients such as sodium methyl cocoyl taurate and polysorbate 20.

TABLE 8

| | wt. % (based on |
|---|---|
| | total composition) |
| alkyl polyglucoside(s) | 12-40 |
| co-solvent or carrier for water | 5-15 |
| pH balancer | 0.1- |
| chelating agent | 0.05-1 |
| humectant | 0.5-3 |
| antimicrobial/preservative(s) | 0.05-1 |
| first solubilizing and clarifying agent | 0.5-6 |
| second solubilizing and clarifying agent (optional) | 0-2 |
| water (balance) | 35-65 |

TABLE 9

| | wt. % (based on |
|---|---|
| | total composition) |
| alkyl polyglucoside(s) | 12-40 |
| co-solvent or carrier for water | 5-15 |
| pH balancer | 0.1-2 |
| chelating agent | 0.05-1 |
| humectant | 0.5-3 |
| antimicrobial/preservative(s) | 0.05-1 |
| first solubilizing and clarifying agent | 0.5-8 |
| second solubilizing and clarifying agent | 0.1-2 |
| water (balance) | 35-65 |

Interestingly, in some compositions/formulations of TABLE 8 and/or TABLE 9, in which Plantaren® 1200 N UP was utilized, the compositions and/or formulations were effective against lice, while less effective or not as effective against fleas. In some compositions and/or formulations of TABLE 8 and/or TABLE 9, when Plantaren® 2000 was utilized, the compositions and/or formulations were effective against fleas, while less effective or not as effective against lice. Thus, compositions and/or formulations herein may be tailored, and made specifically for efficacy and anti-parasitic activity against one desired parasite, or against a combination of desired parasites.

Testing of compositions of TABLES 8 and 9 were performed on live insects, freshly removed from a head of a subject. The lice were placed in cups, generally about 10 lice per cup, and then exposed to a composition and/or formulation for a specified time by adding the composition and/or formulation over the insect. Each test was performed with at least 5 replicates. An alternative shampoo formulation, one not containing any APG, was utilized as a positive control. After the specified time, insects from a cup were poured into a specially sized strainer that captured insects and/or eggs. The strainer was carefully rinsed and then insects were carefully transferred to a petri dish for observation, generally up to two hours total, while noting any change every 15 minutes. All compositions tested performed better than positive control, and, when prepared in final form, are considered environmentally friendly or “green” products. Importantly, when said composition and/or formulation was applied to hair and/or skin, the hair and/or skin was clean, shiny and supple. Working samples of a ready-to-use shampoo form and a shampoo mousse form for hair and/or skin remained stable with storage, maintained split suppression and handleability even with APG at high concentrations (higher than expected to remain stable) while in a lower pH (neutral or acidic).

Further examples of working embodiments of TABLE 8 and/or TABLE 9 are in TABLE 10 and TABLE 11, which also remained stable with storage, maintaining split suppression and handleability with APG at high concentrations (higher than expected to remain stable) and in a lower pH (neutral or acidic). For compositions and/or formulations of TABLE 10, at least one composition/formulation utilizes Plantaren® 1200 N UP for the lauryl glucoside (APG); Pluracare® E 400 NF for the polyethylene glycol (co-solvent or carrier for water); Dissolvine® GL-47-S for the GLDA (chelating agent); glycerin from vegetable (humectant); Argan-o-cide or phenoxyethanol, in combination with caprylyl glycol and isopentyldiol (antimicrobial/preservative); Eumulgin® SML-20 for the polysorbate (first SAC agent), and decyl glucoside or coco glucoside (one or in any combination may also be a SAC agent); citric acid is used as PH balancer. In certain useful compositions and/or formulations, APG is between about 28% and about 32%, polyethylene glycol is between about 9% and about 13%, GLDA is between about 0.1% and about 0.8%, glycerin is between about 1% and about 2%, the combination (caprylyl glycol, phenoxyethanol, and isopentyldiol) is between about 0.1% and about 0.22%, polysorbate is between about 3% and about 5% (10-A) or between about 2% and 4% (10-B), and the alkyl taurate amide and/or taurate salt or decyl glucoside or coco glucoside is between about 0.5% and about 1.5% (10-B).

TABLE 10

| | 10-A | 10-B |
|---|---|---|
| | wt. % (based on total composition) | |
| lauryl glucoside | 20-35 | 20-35 |
| polyethylene glycol | 5-15 | 5-15 |
| citric acid | 0.1-1 | 0.1-1 |
| glutamic acid, N,N,-diacetic acid, | 0.05-1 | 0.05-1 |
| tetrasodium salt (GLDA) | | |
| glycerin | 0.5-3 | 0.5-3 |
| combination: caprylyl glycol, | 0.05-1 | 0.05-1 |
| phenoxyethanol, isopentyldiol | | |
| polysorbate | 0.5-7 | 0.5-5 |
| alkyl taurate amide and/or taurate | 0 | 0.1-2 |
| salt or decyl glucoside | | |
| filtered water | 45-65 | 45-65 |

It is noted that for any composition and/or formulation of TABLE 1-11, a useful source for the APG may be, but is not limited to, Plantaren® or Plantacare® or Plantapon®. Moreover, the APG may be any one or more of APGs described above, such as, but not limited to lauryl glucoside; a C12 alkyl glucoside; a C10-C14 alkyl glucoside; a C10-C16 alkyl glucoside; a C12-C14 alkyl glucoside; coco glucoside that is a coconut based glucoside with any combination of C8-C14 alkyl glucosides, wherein greater than 50% of total alkyl glucosides are one or more of C12 alkyl glucosides and C14 alkyl glucosides; a coconut based glucoside with about 40% C8-C10 alkyl glucosides and about 60% C12-C16 alkyl glucosides; dodecyl D-glucoside having a combination of about 40% C8-C10 alkyl glucosides and about 60% C12-C14 alkyl glucosides; D-glucopyranose, oligomeric, C10-C16-alkyl glycoside; a glucopyranose, oligomeric, a coco glucoside (in quantity or concentration: C8-C10 alkyl chain<C12-C14 alkyl chain); dodecyl D-glucoside; decyl glucoside with predominantly C8-C14 alkyl glucosides, wherein C8 and C10 alkyl glucosides are greater in quantity than C12 and C14 alkyl glucosides; and combinations thereof.

Looking at TABLE 11, at least one form utilizes Plantaren® (e.g., Plantaren® 1200 N UP) as the APG; Pluracare® E 400 NF PEG-FLEX as the polyethylene glycol (co-solvent or carrier for water); Dissolvine® GL-47-S as the GLDA (chelating agent); glycerin from vegetable (humectant); Argan-o-cide or phenoxyethanol in combination with caprylyl glycol and isopentyldiol (antimicrobial/preservative); Eumulgin® SML-20 as the polysorbate (SAC agent; noting that coco betaine, coco glucoside, and sodium methyl cocoyl taurate were also tested individually); and citric acid, adjusted Q.S. to desired pH (e.g., less than pH 7 to about pH 6), with water as needed (to achieve 100%). In certain useful compositions/formulations, APG is between about 28% and about 32%, polyethylene glycol is between about 9% and about 13%, GLDA is between about 0.1% and about 0.7%, glycerin is between about 0.5% and about 2%, the combination (caprylyl glycol, phenoxyethanol, and isopentyldiol) is between about 0.25% and about 0.9%, and polysorbate is between about 3% and about 5%.

TABLE 11

| | 11-A | 11-B |
|---|---|---|
| | wt. % | |
| alkyl polyglucoside(s) | 25-35 | 28-38 |
| polyethylene glycol | 8-14 | 9-15 |
| citric acid (50% solution) to pH | Q.S. | Q.S. |
| between 6.2 and 7.4 | | |
| tetrasodium N,N-bis(carboxylatomethyl)- | 0.05-1 | 0.05-1 |
| L-glutamate (GLDA) | | |
| glycerin | 0.5-2 | 0.5-2 |
| combination: caprylyl glycol, | 0.1-1 | 0.1-1 |
| phenoxyethanol, isopentyldiol | | |
| polysorbate | 1-6 | 2-7 |
| filtered or distilled water | Q.S. | Q.S. |

Working compositions and/or formulations from the tables have no added color, no animal ingredients, no pesticides, and all components (ingredients) are considered cruelty free and non-irritant to skin or hair. Stability and freeze/thaw tests show that working compositions are stable for many months and more than 1 year, remaining as a clear liquid (or only slightly opaque liquid) having viscosity greater than water and more like an oil. Any working composition from the tables may be provided as a shampoo and/or mousse and/or spray and/or spritz. With addition of a thickener, any such composition may be provided as a cream and/or balm and/or paste. And, any working composition may be modified to include one or more of: fragrance (e.g., one or more insect repelling essential oils), one or more thickeners (for providing as paste, balm or cream), and/or one or more enzymes in amounts described (or in TABLE 1).

Compositions and/or formulations herein are considered environmentally friendly or “green” products when in a final form, and, for utility as an insecticide or anti-parasitic and/or for cleaning and conditioning, they are stable, remaining blended and homogenous and maintain clarity when stored (at least three months at room temperature) without any phase separation or release (e.g., no bad odors).

When tested, each tested composition performed as an anti-parasitic by killing and/or knocking down one or more parasites from a group of lice, fleas, biting flies, bedbugs, mites and/or ticks (not all data shown). Unexpectedly, when any of a herein composition and/or formulation was applied to hair and/or skin, the hair and/or skin was clean (removal of unwanted sebum, removal of dandruff, removal of environmental dirt and dust, and removal of residues from hair and/or skin), as well as shiny and supple. Additionally, all said compositions whether ready-to-use or concentrate were stable after storage (several months, up to 3 years) while maintaining split suppression and handleability, even with APG at high concentrations (higher than expected to remain stable) and in a lower pH (neutral or acidic).

Compositions and/or formulations herein may be stored in any container sufficient for providing a product herein for hair and/or skin. Non-limiting examples include tube, jar, bottle, tin, vial, any of which may be squeezable and/or may contain a pump or spray unit or other means for delivery. As a kit, said container having sufficient product for at least one application may be provided with a comb and/or brush and/or wipes and/or means for delivery and/or means for removal (at least in part) with suitable instructions for use and appropriate packaging.

For use, a described composition and/or formulation (preferably ready-to-use or properly diluted) is shaken well before use, applied generously to hair and/or skin that is dry, and then massaged into hair or onto scalp and/or skin. All surfaces suspected of containing insects or an infestation from said insects should be well covered without leaving any dry or un-applied spots or areas. The composition and/or formulation is left undisturbed for a period of time. The period may be 10 minutes. The period may be 15 minutes. The period may be 20 minutes. The period may be 30 minutes. The period may be 45 minutes. The period may be 1 hour. Generally, the period does not need to be more than one or two hours. An additional massage step may be performed, which if requiring moistening of the hair and/or scalp, only finger tips should be lightly wetted so as not to dilute the prepared composition and/or formulation. After the period is complete, the composition and/or formulation is then at least partially removed by any one or any combination of means for removing at least a portion of, or most, or nearly all of the composition/formulation, e.g., utilizing water and/or is wiped-off and/or combed out and/or brushed out (each provided as effective and representative, non-limiting examples). The skin and/or hair may be conditioned after the at least partial removal of the composition and/or formulation (or product therefrom); however, no further conditioning is necessary. An additional step of combing the hair and/or skin is helpful to ensure removal of dead insects, eggs, nits, etc. An application may be repeated, as needed, or the process is continued as needed for conditioning and/or cleansing and/or prophylaxis against re-infestation of said insects for as long as is desired or as needed.

One or more components of a composition and/or formulation described herein are considered safe for use in the U.S. (having GRAS status), UK, Europe, Asia, India, Australia, South America, Central America, Mexico, Africa, Middle East, and/or Japan, and/or elsewhere outside the U.S. In some embodiments, each component of a composition and/or formulation described herein is considered safe and has GRAS status (in the U.S.) or is in conformance with safe use outside the U.S.

The above description and examples demonstrate that compositions and/or formulations herein may be so prepared for multi-functional activity, which unexpectedly includes anti-parasitic (killing, inhibition and/or knock down) activity, prophylactic activity (against re-infestation), cleansing activity and conditioning activity. A composition and/or formulation herein may be manipulated to achieve a level of activity that is more or less selective or expansive against different parasites; the degree of killing and/or knock-down effect may be altered by substitution of one or more APGs with another. Anti-parasitic activity found with compositions and/or formulations herein change what was previously understood regarding the utilization of an APG.

Although representative processes and compositions have been described in detail herein, those skilled in the art will recognize that various substitutions and modifications for preparation of the new lauric acid derivatives may be made without departing from the scope and spirit of what is described and defined by the appended claims.

1. A composition for treatment against one or more parasitic insects on one or more of hair and skin, the composition comprising a uniform blend comprising:
an alkyl polyglucoside as an active ingredient and a primary surfactant in the composition, wherein the alkyl polyglucoside is in an amount greater than 15% and less than 45%, based on weight of a total composition;
filtered water;
a co-solvent for the filtered water in an amount between about 6% and about 15%, based on weight of the total composition;
a humectant in an amount between about 0.5% and about 3%, based on weight of the total composition;
a chelating agent in an amount up to about 1%, based on weight of the total composition;
a pH balancing agent;
a solubilizing and clarifying agent in an amount between about 2% and about 8%, based on weight of the total composition; and
an antimicrobial agent for a hair or skin composition, the antimicrobial agent for protecting the composition against any one or more of decay, discoloration, oxidation and bacterial degradation, wherein the antimicrobial agent for the hair or skin composition is in an amount up to about 1%, based on weight of the total composition.

2. The composition of claim 1, wherein the alkyl polyglucoside is selected from one or more of: (a) lauryl glucoside; (b) a C12-C14 alkyl glucoside; (c) a C10-C14 alkyl glucoside; (d) a coco glucoside that is a coconut based glucoside with any combination of C8-C14 alkyl glucosides, wherein greater than 50% of total alkyl glucosides are one or more of C12 alkyl glucosides and C14 alkyl glucosides; (e) a coconut based glucoside with about 40% C8-C10 alkyl glucosides and about 60% C12-C16 alkyl glucosides; (f) a dodecyl D-glucoside having a combination of about 40% C8-C10 alkyl glucosides and about 60% C12-C14 alkyl glucosides; (g) a coco glucoside with C8-C10 alkyl chains<C12-C14 alkyl chains; (h) a decyl glucoside with about 60% C8-C10 and about 40% C12-C14; (i) a decyl glucoside with C8-C10 alkyl chains>C12-C14 alkyl chains; (j) a predominantly C8-C14 alkyl glucoside; (k) a predominantly C12-C16 alkyl glucoside; (l) a predominantly C8-C16 alkyl glucoside; (m) a decyl glucoside with predominantly, in quantity or concentration, C8-C10 alkyl chains>C12-C14 alkyl chains; (n) a coco glucoside with predominantly, in quantity or concentration, C8-C10 alkyl chains<C12-C14 alkyl chains; (o) a dodecyl D-glucoside; (p) a predominantly C10-C16 alkyl glucoside; (q) a predominantly C10-C14 alkyl glucoside; (r) a predominantly C10-C16 alkyl glucoside; and (s) alkyl glucoside with C6-C20 alkyl chains with predominantly C8 to C14 alkyl chains, wherein greater than 50% of total alkyl glucosides are one or more of C10 alkyl glucosides, C12 alkyl glucosides and C14 alkyl glucosides; and any combination thereof.

3. The composition of claim 1, wherein the alkyl polyglucoside in a ready to use formulation is in an amount greater than about 20 wt. % and up to about 35 wt. %, based on weight of the total composition.

4. The composition of claim 1, wherein the humectant is selected from one or more from a group consisting of one or more of glycerin, glyceryl stearate, triglyceryl stearate, sorbitol, butylene glycol, diethylene glycol, 1,3-propanediol, caprylyl glycol, glycine betaine, grape seed oil, lanolin, acetylated lanolin, honey, pyroglutamic acid, and tristearin.

5. The composition of claim 1, wherein the composition is further utilized to clean and condition the one or more of hair and skin.

6. The composition of claim 1, wherein the chelating agent is selected from one or more from a group consisting of disodium ethylene diamine tetraacetic acid (EDTA), disodium EDTA-copper, HEDTA, oxalic acid, potassium citrate, sodium citrate, tetrahydroxypropyl ethylenediamine, sodium oxalate, TEA-EDTA, tetrasodium EDTA, trisodium EDTA, trisodium HEDTA, nitrilotriacetic acid (NTA), iminodisuccinate (IDS), citric acid, sodium gluconate, ethylenediamine-N,-N-disuccinic acid (EDDA), ethylenediaminedisuccinic acid (EDDS), L-glutamic acid N,N-diacetic acid tetrasodium salt (GLDA), methyl glycinediacetic acid (MGDA), ethylenediamine N,N′-diglutaric acid (EDDG), ethylenediamine-N,N-diamalonic acid (EDDM), 2-hydroxyethyliminodiacetic acid (HEIDA), 3-hydroxy-2,2-iminodisuccinic acid (HIDS), and pyridine-2,6-dicarboxylic acid (PDA).

7. The composition of claim 1, wherein the composition on the one or more of hair and skin is effective as an insecticide against the one or more parasitic insects when the composition is provided on the one or more of hair and skin and remains undisturbed for a period of time being from about 10 minutes to not more than two hours before removal, wherein the one or more parasitic insects is selected from one or more from a group consisting of lice, fleas, bedbugs, mites, scabies, and ticks.

8. The composition of claim 1, wherein the co-solvent for water is selected from one or more from a group consisting of glycol, a modified glycol, and polyethylene glycol.

9. The composition of claim 1, wherein the composition on the one or more of hair and skin is effective as an ovicide against eggs of the one or more parasitic insects when the composition is provided on the one or more of hair and skin and remains undisturbed for a period of time being from about 10 minutes to not more than two hours before removal, wherein the one or more parasitic insects is selected from one or more from a group consisting of lice, fleas, bedbugs, mites, scabies, and ticks.

10. The composition of claim 1, wherein the pH balancing agent is selected from one or more from a group consisting of citric acid, salicylic acid, and lactic acid, and is in an amount sufficient to adjust pH of the composition to a pH of about 8 or less and greater than 6.

11. The composition of claim 1, wherein the solubilizing and clarifying agent is selected from one or more from a group consisting of polysorbate 20, polysorbate 80, a fatty acid alkanolamide, lauric monoethanolamide, diethanolamide, cocamide, caprylyl/capryl glucoside, decyl glucoside, polyethylene glycol (PEG)-50 shea butter, olive oil PEG-7 esters, coco betaine and sulfated castor oil.

12. The composition of claim 1, wherein the solubilizing and clarifying agent further comprises a solubilizing and clarifying agent selected from one or more from a group consisting of fatty alkanolamide, a alkyl taurate amide, and a taurate salt, wherein the alkyl taurate amide and the taurate salt is one or more from a group consisting of sodium methyl cocoyl taurate, potassium taurate, sodium methyl taurate, sodium taurate, sodium lauroyl taurate, calcium lauroyl taurate, sodium methyl lauroyl taurate, magnesium methyl cocoyl taurate, potassium methyl cocoyl taurate, sodium methyl oleoyl taurate, sodium methyl myristoyl taurate, sodium methyl palmitoyl taurate, sodium methyl stearoyl taurate, sodium taurine cococyl methyl taurate, sodium n-isosearoyl methyltaurate, sodium methyltaurine cococyl methyltaurate, sodium taurine cocoyl methyltaurate, sodium cocoyl glutamate, sodium laureth-13 carboxylate, sodium cocoyl isethionate, alkyl methyl glucamide, lauroyl methyl glucamide, cocoyl methyl glucamide, myristoyl methyl glucamide, capryloyl/caporyl methyl glucamide, and sodium cocoyl glycinate.

13. The composition of claim 1, wherein the antimicrobial agent for the hair or skin composition is one or more from a group consisting of glycol ether, caprylyl glycol, isothiazolinones, citricidals, isopentyldiol, phenoxyethanol, stannic chloride, EDTA, benzoic acid/sodium benzoate, sorbic acid/potassium sorbate, levulinic acid, anisic acid, benzalkonium chloride, triclosan, and ethyl lauroyl arginate.

14. The composition of claim 1, wherein the composition for treatment is provided to the one or more of hair and skin, is massaged on the one or more hair and skin, and remains undisturbed for a period of time before removal from the one or more of hair and skin, the period of time being from about 10 minutes and not more than about two hours.

15. The composition of claim 1, wherein the filtered water is adjusted to bring the composition to 100%, the adjustment in a ready to use composition being from between about 35% and about 65%, based on weight of the total composition.

16. A composition for utilization against one or more parasitic insects on one or more of hair and skin, the composition comprising a homogenous blend comprising:
an alkyl polyglucoside as an active ingredient and a primary surfactant in the composition, wherein the alkyl polyglucoside is selected from at least one of: (a) lauryl glucoside; (b) a C12-C14 alkyl glucoside; (c) a C10-C14 alkyl glucoside; (d) a coco glucoside that is a coconut based glucoside with any combination of C8-C14 alkyl glucosides, wherein greater than 50% of total alkyl glucosides are one or more of C12 alkyl glucosides and C14 alkyl glucosides; (e) a coconut based glucoside with about 40% C8-C10 alkyl glucosides and about 60% C12-C16 alkyl glucosides; (f) a dodecyl D-glucoside having a combination of about 40% C8-C10 alkyl glucosides and about 60% C12-C14 alkyl glucosides; (g) a coco glucoside with C8-C10 alkyl chains<C12-C14 alkyl chains; (h) a decyl glucoside with about 60% C8-C10 and about 40% C12-C14; (i) a decyl glucoside with C8-C10 alkyl chains>C12-C14 alkyl chains; (j) a predominantly C8-C14 alkyl glucoside; (k) a predominantly C12-C16 alkyl glucoside; (l) a predominantly C8-C16 alkyl glucoside; (m) a decyl glucoside with predominantly, in quantity or concentration, C8-C10 alkyl chains>C12-C14 alkyl chains; (n) a coco glucoside with predominantly, in quantity or concentration, C8-C10 alkyl chains<C12-C14 alkyl chains; (o) a dodecyl D-glucoside; (p) a predominantly C10-C16 alkyl glucoside; (q) a predominantly C10-C14 alkyl glucoside; (r) a predominantly C10-C16 alkyl glucoside; and (s) alkyl glucoside with C6-C20 alkyl chains with predominantly C8 to C14 alkyl chains, wherein greater than 50% of total alkyl glucosides are one or more of C10 alkyl glucosides, C12 alkyl glucosides and C14 alkyl glucosides; and any combination thereof;
filtered water;
a co-solvent for the filtered water, wherein the co-solvent is a polyethylene glycol with a low average number of ethylene glycol units that is at or about 400 ethylene glycol units or less;
a humectant, wherein the humectant is a vegetable glycerin;
a chelating agent, wherein the chelating agent is glutamic acid, N,N-diacetic acid, tetrasodium salt;
a solubilizing and clarifying agent wherein the solubilizing and clarifying agent is selected from one or more from a group consisting of polysorbate 20, polysorbate 80, caprylyl/capryl glucoside, decyl glucoside, polyethylene glycol (PEG)-50 shea butter, olive oil PEG-7 esters, coco betaine and sulfated castor oil; and
two or more antimicrobial agents utilized for a hair or skin composition and for protecting the composition against any one or more of decay, discoloration, oxidation and bacterial degradation, wherein, with the two or more antimicrobial agents, at least one is a preservative, wherein the two or more antimicrobial agents are two or more from a group consisting of caprylyl glycol, phenoxyethanol, and isopentyldiol; and
a pH balancing agent in a sufficient amount to adjust pH of the composition to a pH greater than about 7.0, wherein the pH balancing agent is citric acid.

17. The composition of claim 16, wherein in a ready to use formulation the composition comprises:
the alkyl polyglucoside in an amount that is between about 26 wt. % and about 33 wt. %;
the co-solvent for water in an amount that is between about 9 wt. % and about 13 wt. %;
the chelating agent in an amount that is up to about 1 wt. %;
the humectant in an amount that is between about 0.5 wt. % and about 3 wt. %,
a combination of the two or more antimicrobial agents in an amount that is up to about 2 wt. %;
the solubilizing and clarifying agent in an amount that is between about 2 wt. % and about 8 wt. %; and
the filtered water in an amount that is between about 39 wt. % and about 62 wt. %,
wherein each wt. % is based on weight of a total composition, and
wherein the composition in the ready to use formulation is effective against the one or more parasitic insects on the one or more of hair and skin after being provided to the one or more of hair and skin and remaining undisturbed for a period of time before removal, the period of time being about 10 minutes or more, and not more than about two hours.

18. The composition of claim 17, wherein in the ready to use formulation the composition is one or more of a single use composition and a repeated use composition, the repeated use composition being selected from at least one from a group consisting of: for a repeat utilization against the one or more parasitic insects; and for a repeat utilization as part of a routine for cleansing and conditioning the one or more of hair and skin.

19. The composition of claim 16, wherein the composition further comprises one or more essential oils.

20. The composition of claim 16, wherein the composition further comprises one or more skincare or haircare enzymes, the one or more skincare or haircare enzymes selected from a group of coenzyme Q10, superoxide dismutase, protease, catalase, diacylglyceroltransferase, lysyl hydroxylase, prolyl hydroxylase, peroxidase, and fruit enzyme.
